(19) United States
(12) Patent Application Publication
(10) Pub. No.: US 2007/0197457 A1
(43) Pub. Date: Aug. 23, 2007

(54) Tak1-mediated inhibition of osteogenesis

(76) Inventors: Dan Gazit, Maccabim (IL); Gadi Pelled, Rishon-Lezion (IL); Gadi Turgeman, East Binyamin (IL); Andrea Hoffmann, Hannover (DE); Gerhard Gross, Braunschweig (DE); Claas Wodarczyk, Braunschweig (DE); Kristin Verschueren, Everberg (BE)

Correspondence Address: Martin Moynihan;Prtsi Inc, P O Box 16446, Arlington, VA, 22215, US

(21) Appl. No.: 10/551,717

(22) PCT Filed: Mar. 29, 2004
(86) PCT No.: PCT/IL04/00286

§ 371 Date: Jul. 14, 2006

Related U.S. Application Data
(60) Provisional application No. 60/458,954, filed on Apr. 1, 2003.

(51) Int. Cl. A61K 48/00

(52) U.S. Cl. 514/44

(57) ABSTRACT

This invention is directed to methods, nucleic acids and compositions in TAK1-mediated regulation of SMAD activity. Promotion of TAK1 interaction with MH2 domains in SMADs negatively regulates SMAD biological activity. BMP-mediated SMAD activity is subject to TAK1 effects.

### FIELD OF THE INVENTION

The present invention is directed to methods, nucleic acids, vectors, cells and/or compositions in TAK1-mediated regulation of SMAD activity. This invention also provides methods for promoting or suppressing osteogenesis, methods of treating conditions wherein promoting or suppressing osteogenesis is beneficial, and methods of screening for candidate genes involved in downstream events in BMP-mediated SMAD-signaling resulting in osteogenesis.

### BACKGROUND OF THE INVENTION

TGF-β activated kinase (TAK1) was initially identified as a cytosolic component of mitogen activated protein kinase (MAPK) pathways activated by ligands of the TGF-βand BMP family of secreted factors. TAK1 is a MAP kinase kinase kinase (MAPKKK, MAP3K) activated by the cytokines IL-1 and TNF-α, in addition to TGF-β/BMPs, consisting of roughly 600 amino acids, with an N-terminal kinase-domain of roughly 300 amino acids, which is roughly 30% homologous to catalytic domains of other MAP3Ks (e.g. Raf-1, MEKK-1). TAK1 mediates activation of c-jun N-terminal kinases (JNK), p38-MAPK and NF-KB pathways. However, molecular events in TAK1 involved signaling cascades, in particular early events in the cascade are as yet not well defined.

SMAD proteins are a family of eight intracellular proteins (SMAD1 to SMAD8) whose members transduce signals for TGF-β ligands including the bone morphogenetic proteins (BMPs). SMAD proteins can be classified into three types according to their structure and mechanism of action. The receptor-regulated SMADs (R-SMADs) SMAD 1-3, SMAD5 and SMAD8, are directly phosphorylated and activated by a TGF-β/BMP type I receptor. Another SMAD type, the common mediator SMAD (Co-SMAD; SMAD4), associates with activated R-SMADs, although hetero-oligomeric activated R-SMAD complexes without SMAD4 have also been detected. SMAD complexes accumulate in the nucleus and participate in the regulation of target genes. Regulation is predominantly at the level of complex binding to other transcription factors and transcriptional regulators, however, direct SMAD binding to DNA via SMAD MH1 domain, or a combination of both mechanisms, may participate in the regulation of target gene expression. The third type of SMADs (I-SMADs, SMAD6 and SMAD7) are termed anti-SMADs or inhibitory SMADs owing to their interference with the activation of, and subsequent complex formation by R-SMADs.

### SUMMARY OF THE INVENTION

This invention relates to methods, nucleic acids and compositions in TAK1-mediated regulation of SMAD activity.

In one embodiment, the invention provides a method of a method of diminishing or abrogating SMAD activity comprising the steps of contacting a cell with an agent that stimulates or enhances TAK1 expression, wherein TAK1 interacts with an MH2 domain of a SMAD protein, thereby diminishing or abrogating SMAD activity.

In another embodiment, the invention provides a method of stimulating or enhancing SMAD activity comprising the steps of contacting a cell with an agent that diminishes or abrogates TAK1 interaction with an MH2 domain of a SMAD protein, thereby stimulating or enhancing SMAD activity.

In another embodiment, the invention provides a method of stimulating or enhancing BMP-mediated SMAD activity comprising the steps of contacting a cell with an agent that diminishes or abrogates TAK1 expression or function.

In another embodiment, the invention provides a method of diminishing or abrogating BMP-mediated SMAD activity comprising the steps of contacting a cell with an agent that stimulates or enhances TAK1 expression or function.

In another embodiment, the invention provides a method of enhancing osteogenesis in a subject in need, comprising the steps of contacting a cell with osteogenic potential in said subject with an agent that mitigates or abrogates TAK1 expression or function, thereby enhancing osteogenesis in said subject.

In another embodiment, the invention provides method of enhancing osteogenesis in a subject in need, comprising the steps of (a) genetically engineering a cell with osteogenic potential to be deficient in TAK1 expression or function and (b) administering said engineered cell to said subject in need, thereby enhancing osteogenesis in said subject.

In another embodiment, the invention provides a method of enhancing bone repair in a body of a subject in need comprising the steps of contacting a cell with osteogenic potential in said subject with an agent that mitigates or abrogates TAK1 expression or function, thereby enhancing bone repair in a body of said subject.

In another embodiment, this invention provides for a method of enhancing bone repair in a subject in need, comprising the steps of (a) genetically engineering a cell with osteogenic potential to be deficient in TAK1 expression or function and (b) administering said engineered cell to said subject in need, thereby enhancing bone repair in said subject.

In another embodiment, this invention provides a method of suppressing osteogenesis in a subject in need, comprising the steps of contacting a cell with osteogenic potential in said subject with an agent that stimulates or enhances TAK1 expression or function, thereby suppressing osteogenesis in said subject.

In another embodiment, this invention provides a method for the identification of candidate gene products involved in downstream events in BMP-mediated SMAD activity resulting in osteogenesis, comprising (a) introducing an agent that inhibits or abrogates TAK1 binding to SMAD MH2 domains into a cell with osteogenic potential, (b) culturing a cell with osteogenic potential as in (a), without said agent, (c) separately harvesting RNA from each cell following stimulation of BMP-mediated SMAD-signaling and (d) assessing differential gene expression, wherein differentially expressed genes in (a) as compared to (b) indicates that the gene is involved, in downstream events in BMP-mediated SMAD activity resulting in osteogenesis.

In another embodiment, this invention provides a method for the identification of candidate gene products involved in downstream events in BMP-mediated SMAD activity resulting in osteogenesis, comprising (a) introducing an agent that inhibits or abrogates TAK1 binding to SMAD MH2 domains into a cell with osteogenic potential, (b) culturing a cell with osteogenic potential as in (a), without said agent, (c) separately harvesting RNA from each cell following stimulation of BMP-mediated SMAD-signaling and (d) assessing differential gene expression, wherein differentially expressed genes in (a) as compared to (b) indicates that the gene is involved in downstream events in BMP-mediated SMAD activity resulting in osteogenesis.

In another embodiment, this invention provides for an isolated nucleic acid, wherein said nucleic acid sequence is antisense to the nucleic acid sequence as set forth in SEQ ID Nos: 1 or 2, or a fragment thereof.

In another embodiment, this invention provides for an oligonucleotide of at least 12 bases specifically hybridizable with the isolated nucleic acid sequence as set forth in SEQ ID Nos: 1 or 2.

### BRIEF DESCRIPTION OF THE DRAWINGS

FIG. 1A depicts the full-length murine TAK1 cloned by PCR from total kidney RNA. Murine TAK1 cDNA clones corresponded to the full-length human TAK1b sequence representing an unspliced TAK1 variant, (Genbank accession number: XM_{—}131329). TAK1 splice variants, kinase domain location, TAK1 deletions, constitutively active (ca) and dominant negative (dn) variants used are as illustrated.

FIG. 1B demonstrates the tissue distribution of the two TAK1 splice variant mRNA in mouse tissue as detected by RT-PCR.

FIG. 1C is a schematic representation of wild-type SMADs and SMAD1 domains used for functional studies in HEK293T and murine C3H10T2 cells. MH1 and MH2 are the major conserved SMAD-domains while “L” specifies the linker region located between MH1 and MH2 domains.

FIG. 2 demonstrates TAK1 interaction with R-SMAD1, SMAD5, and with SMAD4. Human embryonic kidney (HEK) 293T cells were transfected with Flag-, Myc- or HA-tagged expression plasmids. The total amount of DNA transfected was adjusted with empty vector where appropriate. After transfection the cells were harvested, lysed and subjected to immunoprecipitation (IP). Cell extracts and immunoprecipitates were analyzed by SDS-PAGE followed by Western analysis, using is the appropriate anti-tag-specific antibodies. SMAD-1 and SMAD-5 co-immunoprecipitated with TAK1(A). SMAD-2, SMAD-3 (B) and SMAD-4 (C) similarly associated with TAK1.

FIG. 3 demonstrates that TAK1 interaction with R-SMAD1 or with I-SMAD6 (and SMAD7) was dependent on the presence of an active kinase domain within TAK1. Human embryonic kidney (HEK) 293T cells were transfected with Flag- or Myc-tagged expression plasmids and cell extracts were processed as in FIG. 2. SMAD6 (panel (A)) and SMAD-7 (panel (B)) associated with TAK1 containing an active kinase domain, in non-stimulated cells. (C) SMAD1 also interacted only with TAK1 harboring an intact kinase domain. Deletions in TAK1 (1-300 (DC) and 301-606 (DN)) affected SMAD binding, indicating that both the TAK1 kinase domain and the remaining moiety were necessary for SMAD interaction.

FIG. 4 demonstrates that SMAD interaction with TAK1 was mediated via a conserved SMAD-MH2 domain. Human embryonic kidney (HEK) 293T cells were transfected with Flag- or Myc-tagged expression plasmids, and cell extracts were treated as described in the legend to FIG. 2. SMAD3 interaction with TAK1 occurs via the MH2 domain of the SMAD protein (A). The SMAD1-MH2 domain was sufficient for TAK1 interaction (B). A 38 amino acid deletion in the carboxy-terminal domain of the SMAD7-MH2 domain (SMAD7 (1-389) mutant) still associated with TAK1, demonstrating that the TAK1 binding domain is within a region of about 160 amino acids (C).

FIG. 5 demonstrates that TAK1 activity stimulated a SMAD1:SMAD4 interaction. Human embryonic kidney (HEK) 293T cells were transfected with HA- or Myc-tagged expression plasmids and cell extracts were processed as described in FIG. 2. X2 indicates that twice the amount of TAK1wt expression vector was added. The SMAD1:SMAD4 interaction was enhanced in the presence of TAK1wt.

FIG. 6 demonstrates that TAK1 negatively interferes with the transactivation potential of the SMAD-MH2 domain. Reporter assays using the SMAD1-SBE with full-length SMAD1 (A) or a GAL4 reporter with the GAL4 DNA binding (GAL4DBD) domain fused to various forms of SMAD proteins (B-C), in the presence of TAK1 variants, were performed as described. The SBE reporter encodes the CAT gene under the control of nine SMAD1-SBEs, while the GAL4 reporter plasmid encodes firefly luciferase under the control of five UAS sites upstream of a minimal promoter. Results are expressed as CAT or luciferase units normalized to b-galactosidase activity (which results from co-transfected RSV-LTR promoter-E. coli LacZ plasmid). TAK1 interfered with SMAD1-mediated transcription in a dose-dependent fashion (A). TAK1 interfered with the SMAD1-dependent transactivation potential in a dose-dependent fashion (B). TAK1 mediated interference with SMAD1 transactivation required the presence of a complete SMAD-MH2-domain (C). Numbers for TAK1-variants reflect the amount of expression vector added (in nanograms).

FIG. 7 demonstrates TAK1 interference with nucleo-cytoplasmic shuttling of SMADs. Flag-tagged SMAD1 and SMAD3 were visualized by indirect immunofluorescence using an anti-Flag antibody after transfection of the appropriate expression vectors into HEK293T cells Active TAK1 interfered with nucleo-cytoplasmic shuttling as evidenced by SMAD retention within the cytoplasm.

FIG. 8 demonstrates TAK1 activity interfered with BMP-dependent differentiation potential of murine mesenchymal progenitors (C3H10T½). Expression of a TAK1 mRNA splice-variant in murine mesenchymal progenitors C3H10T½ was detected by PCR (A). Western analysis using a polyclonal anti-TAK1 antibody revealed expression levels of recombinantly expressed TAK1 proteins in stably transfected C3H10T½-BMP2 cell lines, (endogenous TAK1 is not visualized under these conditions) (B). TAK1ca contains a deletion of 22 amino acids and is, therefore, smaller than wild-type (wt) and dominant-negative (dn;W63K) TAK1 forms (arrows). The synthesis level for TAK1ca is lower due to its cytotoxicity, allowing expression of low to moderate levels, only. Histological analysis of C3H10T½wt and C3H10T½-BMP2 cells expressing TAK1 variants at 10 days post-confluence (C). While C3H10T½-BMP2 and C3H10T½-BMP2/TAK1dn grow in multilayers (upper panels) C3H10T½-BMP2/TAK1ca and C3H10T½-BMP2/TAK1wt cells grow in monolayer similar to C3H10T½wt (lower panels). C3H10T½-BMP2/TAK1dn cells displayed enhanced osteoblast-like cell formation in comparison to C3H10T½-BMP2 cells, as evidenced by alkaline phosphatase staining. TAK1wt and TAK1ca interfered with osteoblast-like cell formation in C3H10T½-BMP2 cells. Semi-quantitative PCR analysis of osteo-/chondrogenic marker gene expression demonstrated reduced expression of osteogenic marker genes (PTH/PTHrP receptor; osteocalcin) but not chondrogenic marker genes (collagen II) in TAK1wt and TAK1ca samples (D).

FIG. 9 demonstrates TAK1 negligibly influences cell cycle progression in C3H10T½-BMP2 cells. Exponentially growing cells were harvested at indicated times prior to and following cellular confluence (confluence=day 0). C3H10T½-BMP2 cells in the presence of known apoptosis-inducing agent CDDP (25 μM) arrested in G2-M, while the addition of known apoptosis-inducing agent etoposide (10 μM) caused measurable apoptosis, as visualized by a significant subG0-G1 peak. The shoulder in the G0-G1 peak indicated the presence of cells in early apoptosis, as well (arrows) (A). Dominant negative, wild-type and active TAK1 did not induce apoptosis in C3H10T½-BMP2 cells (C3H10T½-BMP2/TAK1dn, C3H10T½-BMP2/TAK1wt or C3H10T½-BMP2/TAK1ca) nor significantly change proportions of cells in G0/G1 versus G2/M phases (X-axis indicates relative DNA concentration; Y-axis indicates cell numbers, 3 independent experiments conducted, representative results shown) (B-D).

### DETAILED DESCRIPTION OF TH INVENTION

This invention provides novel nucleic acids, vectors, compositions and methods for regulating SMAD expression or activity via TAK1, and therapeutic applications arising from their utilization.

SMADs are a family of intracellular signaling proteins, which transduce signals thereby mediating a wide range of biological processes, including regulation of cell proliferation, differentiation, recognition, and death, and thus play a major role in developmental processes, tissue recycling, and repair. Effects that result in enhanced or diminished SMAD protein expression, or otherwise limit or enhance SMAD signal transduction will have substantial impact on biological processes.

TGF-β activated kinase (TAK1) is a MAP3K activated by ligands of the TGF-β and BMP family of secreted factors and by the cytokines IL-1 and TNF-α. Like the SMADs, TAK1 is involved in signaling cascades, with a definitive role in negative regulation of SMAD biological activity.

Direct interaction of TAK1 with SMADs is demonstrated in Example 2, hereinbelow, with intact TAK1 kinase activity being a prerequisite for SMAD binding. The conserved SMAD MH2 domain is the minimal domain required for the TAK1 interaction (Example 3).

TAK1 interaction with SMAD via its MH2 domain results in reduction of SMAD activity, in terms of its transactivation potential and transcription following receptor-mediated activation (Example 4). Thus, in its native form, TAK1 negatively-regulates SMAD biological activity.

By the term “biological activity” or “SMAD activity” or “SMAD biological activity”, it is meant, in one embodiment, to include all intracellular activity mediated via SMAD, including downstream effects in a given signal cascade, which are halted or altered as, a result of SMAD absence or diminution in “concentration. Any function attributable to SMAD involvement is considered encompassed by the term.

The term “TAK1” as used herein is meant, in one embodiment, to denote any TGF-βactivated kinase involved in signaling cascades, intracellularly, which in its native state interacts with a SMAD. In one embodiment, the TAK1 protein corresponds to, or in another embodiment is homologous to, Genbank Accession numbers O43318, NP_{—}663304, NP_{—}663305 or NP_{—}663306, or is homologous to Genbank Accession numbers NM_{—}172688, NM_{—}079356.

In another embodiment, the TAK1 protein is encoded by a nucleic acid sequence that corresponds to, or in another embodiment is homologous to, Genbank Accession numbers NM_{—}145333, NM_{—}145332, NM_{—}145331, NM_{—}003188 or is homologous to Genbank Accession number NP_{—}524080.

As used herein, the term “correspond to” or “correspondance” in reference to a protein or nucleic acid refers to an amino acid or nucleic acid sequence, respectively, that is identical to the referenced Sequence. The terms “homology”, “homologue” or “homologous”, in any instance, indicate that the nucleic acid or amino acid sequence referred to, exhibits, in one embodiment at least 70% correspondence with the indicated sequence. In another embodiment, the nucleic acid or amino acid sequence exhibits at least 75% correspondence with the indicated sequence. In another embodiment, the nucleic acid or amino acid sequence exhibits at least 80% correspondence with the indicated sequence. In another embodiment, the nucleic acid or amino acid sequence exhibits at least 85% correspondence with the indicated sequence. In another embodiment, the nucleic acid or amino acid sequence exhibits at least 90% correspondence with the indicated sequence. In another embodiment, the nucleic acid or amino acid sequence exhibits at least 95% or more correspondence with the indicated sequence. In another embodiment, the nucleic acid or amino acid sequence exhibits 95%-100% correspondence with the indicated sequence.

Protein and/or peptide homology for any peptide sequence listed herein may be determined by immunoblot analysis, or via computer algorithm analysis of amino, acid sequences, utilizing any of a number of software packages available, via methods well known to one skilled in the art. Some of these packages may include the FASTA, BLAST, MPsrch or Scanps packages, and may employ the use of the Smith and Waterman algorithms, and/or global/local or BLOCKS alignments for analysis, for example.

Nucleic acid sequence homology may be determined by any number of computer algorithms available and well known to those skilled in the art, for example, the Smith-Waterman algorithm, utilized in analyzing sequence alignment protocols, as in for example, the GAP, BESTFIT, FASTA and TFASTA programs in the Wisconsin Genetics Software Package release 7.0, Genetics Computer Group, 575 Science Dr., Madison, Wis.). For example, the percent homology between two nucleotide sequences may be determined using the GAP program in the GCG software package, using a NWS gap DNA CMP matrix and a gap weight of 40, 50, 60, 70, or 80 and a length weight of 1, 2, 3, 4, 5, or 6.

Nucleic acid sequence homology may be determined by hybridization to a reference sequence under highly stringent (0.2×SSC at 65° C.), stringent (e.g. 4×SSC at 65 C or 50% formamide and 4×SSC at 42° C.), or relaxed (4×SSC at 50° C. or 30-40% formamide and 4×SSC at 42° C.) conditions.

By the term “SMAD” or “SMADs” as used herein, it is meant to include, in one embodiment, any family member of the SMAD intracellular signaling proteins, which transduce signals for TGF-β, regardless of species. SMADs include but are not limited to SMAD-1, SMAD-2, SMAD-3, SMAD-4, SMAD-5, SMAD-6, SMAD-7 or SMAD-8.

As contemplated herein, the nucleic acid, which encodes for a SMAD protein may correspond to, or in another embodiment, be homologous to Genbank Accession Numbers: NM 005905, NT 016606, NM 008539, AF 067727, NM 010754, AB 071949, AH006488, AF 056001, AB 008192, NM 005902, NM 016769, NT 010265, NT 033905, AB 043547, AB 010954, AF 056002, NT 016714, AH005750, AH 005612, MN 008541, AB043547, AH008461, AF037469, AF 043640, AH011391, AH008243, AJ000550, AF175408, MN 139972, MN 005905 or MN 19483.

As contemplated herein, the SMAD amino acid sequence may correspond to, or in another embodiment, be homologous to, Genbank Accession Numbers: Q92940, Q17796, Q99717, O43541, O35253, Q15797, BAA22032, AAB94137, S68987, AAC50790, AAB06852, O15105 or Q15796. In another embodiment, the SMAD amino acid sequence may be homologous to Genbank Accession Numbers: AAN85445, JE0341, P97454, Q62432, P97588, O70436.

In one embodiment, the invention is directed to a method of diminishing or abrogating SMAD activity comprising the steps of contacting a cell with an agent that stimulates or enhances TAK1 expression, wherein TAK1 interacts with an MH2 domain of a SMAD protein, thereby diminishing or abrogating SMAD activity.

The terms “diminishing/es”, “mitigating/es”, “down-regulating/es” or “down-modulating/es” are to be considered synonymous, and meant to include, in one embodiment, quantitative or qualitative reduction. Signaling molecules typically are active in exquisitely low intracellular concentrations, and hence minute alterations in these concentrations may result in a biologically observable effect.

By diminishing SMAD activity, we mean interference with SMAD intracellular function. Such interference, in one embodiment, may be via physical prevention of SMAD interaction with accessory proteins. Such inhibition of SMAD intracellular function may be carried out with minute amounts of TAK1.

SMAD proteins have been shown to function as transcriptional activators, and as such, two hybrid systems may be utilized to measure changes in SMAD transactivational activity, as a measure of SMAD function, as further exemplified in the Examples section that follows.

SMAD activity may, in another embodiment, be measured as a function of its ability to translocate to the nucleus. This can be measured by any number, of techniques known in the art, such as subcellular fractionation followed by immunoblot analysis of the fractions, or for example, by immuno-cytochemistry, as further exemplified in the Examples section that follows.

Diminishing SMAD activity may also include preventing SMAD expression. Receptor-mediated cellular activation, for example, resulting ordinarily in SMAD transcription may be affected by intracellular TAK1 expression, resulting in diminished SMAD transcription. Such a scenario is also comprised in the present invention.

Changes in SMAD gene expression may be measured by methods well described in the art, such as Northern blot and dot blot analysis, primer extension, RNase protection, RT-PCR, or in-situ hybridization.

The terms “abrogating” or “abrogation” refer to the absence of activity, including mRNA or protein expression.

By the term “stimulate/s” it is meant to include any effect which results in the initiation of production of the molecule/s specified, and can include events resulting in the initiation of mRNA expression, or protein production. The term “enhance/s” refers, in one embodiment, to a heightened, greater production of the molecule specified, including, as above, increased mRNA or protein production.

The term “an agent that stimulates or enhances TAK1 expression”, includes, in one embodiment, any chemical entity that produces an increased expression of TAK1 mRNA or protein. In another embodiment, it includes a chemical entity that provides for the expression of TAK1 mRNA or protein, where previously expression of TAK1 was absent.

Increased production of TAK1 mRNA may be demonstrated via numerous methods well known to one skilled in the art. Some methods include Northern blot and dot blot analysis, primer extension, RNase protection, RT-PCR, or in-situ hybridization (see, for example “Current Protocols in Molecular Biology” Volumes I-III Ausubel, R. M., ed. (1994); Ausubel et al., “Current Protocols in Molecular Biology”, John Wiley and Sons, Baltimore, Md. (1989); Mullis & Faloona, 1987, Methods Enzymol., 155:335, Dassi et al., 1998, Clin. Chem., 44:2416). In another embodiment, measurement of TAK1 protein production is another means of verifying TAK1 expression. Methods for detection of TAK1 protein include Western blot analysis, immunoblot analysis, ELISA, RIA or HPLC, to name a few examples.

Such an agent can comprise nucleic acids, or nucleic acid vectors comprising a TAK1 gene, that when introduced are expressed intracellularly, resulting in expression, or elevated expression of TAK1 mRNA and protein.

The term “nucleic acid” describes a polymer of deoxyribonucleotides (DNA) or ribonucleotides (RNA). The nucleic acid may be isolated from a natural source by cDNA cloning or subtractive hybridization or synthesized manually. The nucleic acid may be synthesized by chemical synthesis, manually by the triester synthetic method or by using an automated DNA synthesizer. The nucleic acid may be synthesized by in vitro amplification [including but not limited to the polymerase chain reaction (PCR)], or by combinations of these procedures from naturally occurring sources, such as cultures of mammalian cells, genomic DNA from such cells or libraries of such DNA. The nucleic acid may be single stranded, or double stranded and may assume any 3 dimensional structure. The nucleic acid sequence may also differ from the published TAK1 sequence, yet still encode for a TAK1 protein with an intact kinase domain, and is to be considered an additional embodiment of the present invention.

The terms “protein”, “polypeptide” and “peptide” are used interchangeably herein when referring to a gene product.

As will be appreciated by one skilled in the art, a fragment or derivative of a nucleic acid sequence or gene that encodes for the TAK1 protein or peptide can still, in one embodiment, function in the same manner as the entire, wild type gene or sequence, in particular if it comprises the kinase domain of TAK1. Likewise, forms of nucleic acid sequences can have variations as compared with the wild type sequence, while the sequence still encodes a protein or peptide, or fragments thereof, that retain their wild type function despite these variations. Proteins, protein fragments, peptides, or derivatives also can experience deviations from the wild type from which still functioning in the same manner as the wild type form. Similarly, derivatives of the genes and products of interest used in the present invention will have the same biological effect on the host as the non-derivatized forms. Examples of such derivatives include but are not limited to dimerized or oligomerized forms of the genes or proteins, as wells as the genes or proteins. Biologically active derivatives and fragments of the genes, DNA sequences, peptides and proteins of the present invention are therefore also within the scope of this invention.

The agent may, in another embodiment, comprise a recombinant vector which comprises at least one nucleic acid sequence encoding the TAK1 protein or variant, analog, fragment, mimetic, mutant or synthetic thereof, and compositions comprising same. The nucleic acid sequence encoding the TAK1 protein or variant may be in single or multi-copy and may be conditionally or constitutively expressed, engineered by methods well known to one skilled in the art [see for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York].

Once TAK1 is subcloned into a particular vector it thereby becomes, in one embodiment, a recombinant vector. To generate the nucleic acid constructs in context of the present invention, the polynucleotide segments encoding TAK1 can be ligated into commercially available expression vector systems suitable for transfecting or transducing mammalian cells and for directing the expression of recombinant products within the transfected or transduced cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter polypeptides.

By “vector” what is meant is, in one embodiment, a nucleic acid construct containing a sequence of interest that has been subcloned within the vector, in this case, the nucleic acid sequence encoding TAK1 or a fragment thereof.

A vector according to the present invention may, according to additional embodiments, further include appropriate selectable markers. The vector may further include an origin of replication, and may be a shuttle vector, which can propagate both in prokaryotic, and in eukaryotic cells, or the vector may be constructed to facilitate its integration within the genome of an organism of choice. The vector according to this aspect of the present invention can be, for example, a plasmid, a bacmid, a phagemid, a cosmid, a phage, a virus or an artificial chromosome.

Protocols for producing recombinant vectors and for introducing the vectors into cells may be accomplished, for example, by: direct DNA uptake techniques, virus, plasmid, linear DNA or liposome mediated transduction, or transfection, magnetoporation methods employing calcium-phosphate mediated and DEAE-dextran mediated methods of introduction, electroporation, direct injection, and receptor-mediated uptake (for further detail see, for example, “Methods in Enzymology” Vol. 1-317, Academic Press, Current Protocols in Molecular Biology, Ausubel F. M. et al. (eds.) Greene Publishing Associates, (1989) and in Molecular Cloning: A Laboratory Manual, 2nd Edition, Sambrook et al. Cold Spring Harbor Laboratory Press, (1989), or other standard laboratory manuals).

Additional suitable commercially available mammalian expression vectors include, but are not limited to, pcDNA3, pcDNA3.1(+/−), pZeoSV2(+/−), pSecTag2, pDisplay, pEF/myc/cyto, pCMV/myc/cyto, pCR3.1, which are available from Invitrogen, pCI which is available from Promega, pBK-RSV and pBK-CMV which are available from Stratagene, pTRES which is available from Clontech, and their derivatives. Linear DNA expression cassettes (LDNA) may be employed as well (Chen Z Y et al. Mol Ther. 3:403-10, 2001).

In another embodiment, the vector is engineered to incorporate a reporter gene. The term “reporter gene”, as used herein, refers to a coding unit whose product is easily assayed (such as, without limitation, luciferase or chloramphenicol transacetylase). A reporter gene can be either a DNA molecule isolated from genomic DNA, which may or may not contain introns, or a complementary DNA (cDNA) prepared using messenger RNA as a template. In either case, the DNA encodes an expression product that is readily measurable, e.g., by biological activity assay, enzyme-linked immunosorbent assay (ELISA) or radioimmunoassay (RIA). Expression products of the reporter genes can be measured using standard methods. Various types of immunoassays such as competitive immunoassays, direct immunoassays and indirect immunoassays may be used as well, in order to detect the reporter gene product.

In addition to recombinant vectors enhancing TAK1 expression, other agents may be employed that function to stimulate TAK1 expression including factors that initiate the TGF-β signaling cascade, that result in production of TAK1, including but not limited to, TGF-β and TGF-β receptor agonists. Any agent that results in production or enhanced production of TAK1, which facilitates TAK1 binding to SMAD MH2 domains is considered to comprise additional embodiments of the present invention.

As used herein, the term “contacting a cell” refers, in one embodiment, to both direct and indirect exposure of a cell to an agent or molecule of the invention. In one embodiment, contacting a cell may comprise direct injection of the cell through any means well known in the art, such as microinjection. It is also envisaged, in another embodiment, that supply to the cell is indirect, such as via provision in a culture medium that surrounds the cell. Any agent of the invention may be administered thus, and comprises an embodiment of the invention.

As used herein, the term “administration”, “administer/ed”, “delivery” or “deliver/ed” refers, in one embodiment, to introduction that may be performed topically (including opthalmically, vaginally, rectally, intranasally), orally, by inhalation, or parenterally, for example by intravenous drip or intraperitoneal, subcutaneous, subdural, intramuscular or intravenous injection, or via an implantable delivery device. Any agent of the invention may be administered thus, and comprises an embodiment of the invention.

It is to be understood that the agents of the invention may be administered, in one embodiment, as part of a pharmaceutical composition. Such a pharmaceutical composition may include the nucleic acids in combination with any standard physiologically and/or pharmaceutically acceptable carriers, which are known in the art. The compositions should be sterile and contain a therapeutically effective amount of the nucleic acids in a unit of weight or volume suitable for administration to a patient. The term “pharmaceutically acceptable” refers to a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials, which are well known in the art.

In another embodiment, the agent administered to stimulate or enhance TAK1 expression may comprise a mutated version of TAK1, which may be administered as a naked nucleic acid molecule, or within an expression vector. Mutations in TAK1 may enhance SMAD interaction, may function to promote uptake within cells, or may provide additional benefits for their introduction, and as such are considered as part of this invention. Mutations of the TAK1 nucleic acid sequence may comprise point mutations, substitutions, insertions or deletion mutations, or induced modifications each of which represent an additional embodiment of the invention. Nucleic Acid sequences of the present invention may comprise single mutations, or multiple mutations, including combinations of the mutations listed herein, each of which is to be considered a separate embodiment of the invention.

According to this aspect of the invention, the mutations serve to enhance TAK1 mediated interaction with MH2 domains of SMAD, thereby negatively regulating SMAD activity.

The addition of low levels of a dominant-negative mutant of TAK1, however, not only reversed the suppressive effects on SMAD activity seen with wild-type and constitutively active TAK1, but enhanced transactivation and transcription (Example 4).

Dominant-negative (dn) mutations introduced into a gene result in loss-of-function for the encoded product, however, they typically retain their proper structure and associate with other cellular components, often with a higher than normal affinity for a cellular component, displacing the wild-type protein. In this case, the TAK1dn mutant competed with endogenous TAK1.

Interfering with TAK1 function provides, in one embodiment, a means of stimulating or enhancing SMAD activity.

In another embodiment, there is provided a method of stimulating or enhancing SMAD activity comprising the steps of contacting a cell with an agent that diminishes or abrogates TAK1 interaction with an MH2 domain of a SMAD protein, thereby stimulating or enhancing SMAD activity.

By the phrase “diminishes or abrogates TAK1 interaction”, it is meant to comprise an ultimate interference with the TAK1-SMAD MH2 interaction. Such an interference may be, in one embodiment, via a physical block of the MH2 SMAD domain, preventing TAK1 interaction, or in another embodiment, a physical block of the cognate binding regions in TAK1, or in another embodiment, a method of diminishing or abrogating TAK1 expression, thereby preventing or limiting its association with SMAD.

In one embodiment, the agent is 5Z-7-oxo-zeaenol, or an agent functionally equivalent thereto.

In another embodiment, the agent is designed to physically block SMAD MH2 interaction with TAK1. The design of a molecule that physically block SMAD MH2 interaction with TAK1 can be accomplished by methods well known in the art. The structure of the SMAD protein has been defined (Wu J W et al., Mol Cell (2001) 8(6): 1277-89). Once the structure of TAK1 is established, by for example, X-Ray crystallographic data, NMR or molecular modeling techniques, then binding sites for either can be ascertained, and the binding sites are filled with a close packed array of generic atoms. A Monte Carlo procedure (D. K. Gehlhaar, et al. “De Novo Design of Enzyme Inhibitors by Monte Carlo Ligand Generation” J. Med. Chem. 1995, 38, 466-472) is used to randomly move, rotate, exchange atom types and/or bond types, and the resulting chemical moieties, representing inhibitors for SMAD MH2 and TAK1 respectively, designed can be tested for their ability to inhibit the SMAD MH2-TAK1 interaction.

Other methods that can be modified to design inhibitors of the TAK1-SMAD MH-2 interaction include: MCSS (Multiple Copy Simultaneous Search)/HOOK (A. Caflish, et al. J. Med. Chem. 1993, 36, 2142-2167; M. B. Eisen, et al. Str. Funct. Genetics 1994, 19, 199-221), LUDI (H.-J. Böhm J. Comp.-Aided Mol. Design 1992, 6, 61-78) GROW (J. B. Moon and W. J. Howe Str. Funct. Genetics 1991, 11, 314-328) CoMFA (Conformational Molecular Field Analysis) (J. J. Kaminski, Adv. Drug Delivery Reviews 1994 14 331-337) and other methods known to one skilled in the art.

It is to be understood that any inhibitor designed to regulate SMAD activity via minimizing or preventing the physical interaction between TAK1 and SMAD MH2, for example by the methods listed above, is to be considered part of the present invention.

TAK1 kinase active forms were able to bind efficiently to SMAD proteins, whereas kinase inactive forms, such as in the TAK1 containing a point mutation K63W, which interferes with the kinase activity of TAK1, resulted in interference with TAK1-SMAD binding, (Example 2).

In other embodiments, inhibitors are designed to interfere with a TAK1 kinase domain, thus preventing TAK1-SMAD MH2 interaction, by any of the means listed herein, including the design of specific inhibitors preventing binding, as above, or the use of molecular techniques that diminish or abrogate expression of kinase encoding nucleotides of TAK1, as described hereinbelow, each of which represents an embodiment of the present invention.

In another embodiment, diminishing or abrogating TAK1 expression is a means of preventing the SMAD MH2-TAK1 interaction, thereby stimulating or enhancing SMAD activity.

Down-regulation of endogenous sequences may be accomplished via various means well known in the art. In one embodiment, antisense RNA may be employed as a means of endogenous sequence inactivation. Exogenous polynucleotide(s) encoding sequences complementary to the endogenous TAK1 mRNA sequences, or fragments thereof are administered, resulting in sequence transcription, and subsequent gene inactivation.

In one embodiment, the TAK1 sequence targeted for gene inactivation corresponds to, or is homologous to SEQ ID No: 1 or 2.

In another embodiment, downregulating TAK1 gene expression is via the use of small interfering RNAs (siRNAs). Duplexes consisting of between 15-, and 30-nucleotide siRNA generated by ribonuclease III cleavage of longer dsRNAs, and by cleavage induced by other enzymes (e.g., “dicer” in D. melanogaster (Baulcombe, D. Nature 409(2001): 295-6 and Caplen, N. J., et al. PNAS. 98(2001): 9742-7) thought to be similar to RNase III, or generated artificially, are the mediators of sequence specific mRNA degradation. Overhanging nucleotides on the 3′ ends of the dsRNA help processing proteins recognize the dsRNA and mediate cleavage of the target mRNA. Duplexes associated with the processing protein form small interfering ribonucleoprotein complexes (siRNPs), possessing endonuclease activity, facilitate cutting of the nucleic acids. The siRNPs that contain antisense-siRNA hybridize to complementary sense mRNA and cleave it, and vice versa. Gene silencing entails antisense siRNA-mediated cleavage of target mRNA transcribed from the gene, preventing sells from translating the target mRNA into a protein (Hammond, S M et al Nature 404: 293-296 (2900); Yang, D et al., Curr. Biol. 10: 1191-1200 (2000); and US Patent Application Serial No. 20020086356).

In another embodiment, disrupting TAK1 gene expression can be accomplished using synthetic oligonucleotides capable of hybridizing with endogenous TAK1 double stranded DNA. A triple helix is formed. Such oligonucleotides may prevent binding of transcription factors to the gene's promoter and therefore inhibit transcription. Alternatively, they may prevent duplex unwinding and, therefore, transcription of genes within the triple helical structure.

In another embodiment, TAK1 down regulation can also be effected via gene knock-out techniques, by practices well known in the art (“Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988)).

In another embodiment, dominant negative TAK1 mutants may be generated. Introduction of the mutant competes for endogenous TAK1 binding SMAD MH2 domains, thereby preventing endogenous TAK1 binding, stimulating or enhancing SMAD activity.

The term “dominant negative” is used herein to refer, in one embodiment, to a non-natural TAK1 such as a TAK1 encoded by a genetically altered TAK1 nucleotide sequence. The subject non-natural TAK1's are functionally and/or structurally related to TAK1 but differ from natural TAK1 by virtue of a common ability to suppress TAK1-SMAD MH2 binding, hence suppress TAK1 ability in negatively regulating SMAD activity, and in a dominant negative manner. TAK1dn, as exemplified herein, is a representative example of a dominant negative suppressor of natural TAK1.

In one embodiment, a TAK1 dominant negative mutant is produced which results in the amino acid substitution K63W in TAK1. In another embodiment, a TAK1 dominant negative mutant is produced which results in the amino acid substitution S192A in TAK1.

It is to be understood that additional dominant negative mutants of TAK1 may be generated, and represent additional embodiments of the present invention, when utilized for the purpose of stimulating or enhancing SMAD activity.

Representative examples of genetically altered TAK1 nucleotide sequences include sequences that have been mutated, deleted, inverted, or otherwise modified to create a dominant negative suppressor construct, which includes portions of the SMAD MH2 cognate binding domain of TAK1 or a truncated form or TAK1, which precludes SMAD MH2 interaction with TAK1.

In one embodiment, the dominant negative suppressor of TAK1 nucleotide sequence is at set forth in SEQ ID Nos: 1 or 2. In another embodiment, the nucleic acid sequence comprises a fragment thereof. TAK1 nucleotide coordinates that may be inhibited, in one embodiment, from interacting with SMAD M12 domains comprise nucleotides encoding for amino acids 43 to 284, which encodes the kinase domain of TAK1.

In one embodiment, TAK1 deletion mutants comprise a nucleotide sequence as follows (SEQ ID No: 1):

ATGTCGACAGCCTCCGCCGCCTCGTCCTCCTCCTCGTCTTCTGCCAGTGA
GATGATCGAAGCGCCGTCGCAGGTCCTGAACTTCGAAGAGATCGACTACA
AGGAGATCGAGGTGGAAGAGGTTGTCGGAAGAGGAGCTTTTGGAGTAGTT
TGCAAAGCTAAGTGGAGAGCAAAAGATGTCGCTATTAAACAGATAGAAAG
TGAGTCTGAGAGGAAGGCTTTCATTGTGGAGCTCCGGCAGTTGTCACGTG
TGAACCATCCTAACATTGTCAAGTTGTATGGAGCCTGCCTGAATCCAGTA
TGTCTTGTGATGGAATATGCAGAGGGGGGCTCATTGTATAATGTGCTGCA
TGGTGCTGAACCATTGCCTTACTACACTGCTGCTCATGCCATGAGCTGGT
GTTTACAGTGTTCCCAAGGAGTGGCTTACCTGCACAGCATGCAGCCCAAA
GCGCTGATTCACAGGGACCTCAAGCCTCCAAACTTGCTGCTGGTTGCAGG
AGGGACAGTTCTAAAAATCTGCGATTTTGGTACAGCTTGTGACATCCAAA
CACACATGACCAATAATAAAGGGAGTGCTGCTTGGATGGCGCCTGAAGTA
TTTGAAGGTAGCAATTACAGTGAAAAGTGTGATGTCTTCAGCTGGGGTAT
TATCCTCTGGGAAGTGATAACACGCCGGAAACCCTTCGATGAGATCGGTG
GCCCAGCTTTCAGAATCATGTGGGCTGTTCATAATGGCACTCGACCACCA
CTGATCAAAAATTTACCTAAGCCCATTGAGAGCTTGATGACACGCTGTTG
GTCTAAGGACCCATCTCAGCGCCCTTCAATGGAGGAAATTGTGAAAATAA
TGACTCACTTGATGCGGTACTTCCCAGGAGCGGATGAGCCGTTACAGTAT
CCTTGTCAGTA

In another embodiment, TAK1 deletion mutants comprise a nucleotide sequence as follows (SEQ ID No: 2).

CTCTGATGAAGGGCAGAGCAACTCAGCCACCAGCACAGGCTCATTCATGG
ACATTGCTTCTACAAATACCAGTAATAAAAGTGACACAAATATGGAACAG
GTTCCTGCCACAAACGACACTATTAAACGCTTGGAGTCAAAACTTTTGAA
AAACCAGGCAAAGCAACAGAGTGAATCTGGACGCCTGAGCTTGGGAGCCT
CTCGTGGGAGCAGTGTGGAGAGCTTGCCCCCCACTTCCGAGGGCAAGAGG
ATGAGTGCTGACATGTCTGAAATAGAAGCCAGGATCGTGGCGACTGCAGC
CTATTCCAAGCCTAAACGGGGCCACCGTAAAACCGCTTCATTTGGCAACA
TTCTGGATGTCCCTGAGATCGTCATATCAGGTAACGGGCAACCAAGGCGT
AGATCCATCCAAGACTTGACTGTTACTGGGACAGAACCTGGTCAGGTGAG
CAGCCGGTCATCCAGCCCTAGTGTCAGAATGATCACTACCTCAGGACCAA
CCTCAGAGAAGCCAGCTCGCAGTCACCCGTGGACCCCTGATGATTCCACA
GATACCAATGGCTCAGATAACTCCATCCCAATGGCGTATCTTACACTGGA
TCACCAGCTACAGCCTCTAGCGCCGTGCCCAAACTCCAAAGAATCCATGG
CAGTGTTCGAACAACATTGTAAAATGGCACAGGAGTATATGAAAGTTCAA
ACCGAAATCGCATTGTTACTACAGAGAAAGCAAGAACTAGTTGCAGAATT
GGACCAGGATGAAAAGGACCAGCAAAATACATCTCGTCTGGTACAGGAAC
ATAAAAAGCTTTTAGATGAAAACAAAAGCCTTTCTACTTATTACCAGCAA
TGCAAAAAACAACTAGAGGTCATCAGAAGCCAACAGCAGAAACGACAAGG
CACTTCATGA

In another embodiment, the TAK1 deletion mutants comprise a nucleotide sequence homologous to the sequence set forth in SEQ ID Nos: 1 or 2.

As used herein, the terms “homology”, “homologue” or “homologous”, in any instance, indicate that the nucleic acid sequence referred to, exhibits, in one embodiment at least 70% correspondence with the indicated sequence. In another embodiment, the nucleic acid sequence exhibits at least 75% correspondence with the indicated sequence. In another embodiment, the nucleic acid sequence exhibits at least 80% correspondence with the indicated sequence. In another embodiment, the nucleic acid sequence exhibits at least 85% correspondence with the indicated sequence. In another embodiment, the nucleic acid sequence exhibits at least 90% correspondence with the indicated sequence. In another embodiment, the nucleic acid sequence exhibits at least 95% or more correspondence with the indicated sequence. In another embodiment, the nucleic acid sequence exhibits 95%-100% correspondence with the indicated sequence.

Nucleic acid sequence homology may be determined by any number of computer algorithms available and well known to those skilled in the art, for example, the Smith-Waterman algorithm, utilized in analyzing sequence alignment protocols, as in for example, the GAP, BESTFIT, FASTA and TFASTA programs in the Wisconsin Genetics Software Package release 7.0, Genetics Computer Group, 575 Science Dr., Madison, Wis.). For example, the percent homology between two nucleotide sequences may be determined using the GAP program in the GCG software package, using a NWS gap DNA CMP, matrix and a gap weight of 40, 50, 60, 70, or 80 and a length weight of 1, 2, 3, 4, 5, or 6.

Nucleic acid sequence homology may be determined by hybridization to a reference sequence under highly stringent (0.2×SSC at 65° C.), stringent (e.g. 4×SSC at 65 C or 50% formamide and 4×SSC at 42° C.), or relaxed (4×SSC at 50° C. or 30-40% formamide and 4×SSC at 42° C.) conditions.

In another embodiment, the invention provides for an isolated nucleic acid as set forth in SEQ ID Nos: 1 or 2.

In another embodiment, the invention provides for an isolated nucleic acid at least 70% homologous to SEQ ID Nos: 1 or 2.

In another embodiment, the invention provides for a nucleic acid sequence, which is antisense to the nucleic acid sequence as set forth in SEQ ID Nos: 1 or 2, or a fragment thereof.

In another embodiment, the invention provides for a vector comprising the isolated nucleic acids of the invention. As described, the vector may further comprise a promoter for regulating transcription of the isolated nucleic acid in sense or antisense orientation, positive and/or negative selection markers for selecting for homologous recombination events.

In another embodiment, the invention provides for a host cell or animal comprising the vectors of the invention. The host cell may be prokaryotic, in one embodiment. In other embodiments the host cell may be eucaryotic, and may be a mesenchymal stem cell, a progenitor cell, an osteoblast, or any cell capable of differentiating into an osteoblast.

In another embodiment, the invention provides for an oligonucleotide that comprises only a portion of the nucleic acid sequences of SEQ ID Nos: 1 or 2, for example a fragment which can be used as a probe or primer or a fragment encompassing a biologically active portion of TAK1.

In one embodiment, the oligonucletide corresponds to:

(SEQ ID No: 3)

TATAGGATCCTCATCACTTGTCATCGTCATCCTTGTAGTCATACTGTAAT
GGCTCATCCG
or

(SEQ ID No: 4)

TATAGAATTCGCCACCATGCCTTGTCAGTACTCTGATGA.

The nucleotide sequence allows for the generation of probes and primers designed for use in identifying and/or cloning other TAK1-like molecules, as well as TAK1 family homologues from other species. The probe/primer typically comprises a substantially purified oligonucleotide.

According to this aspect of the invention, in one embodiment, the oligonucleotide will comprise at least 12 or 15, or in another embodiment, at least 20 or 25, or in another embodiment, at least 30, or in another embodiment, at least 35, or in another embodiment, at least 40, or in another embodiment, at least 45, or in another embodiment, at least 50, or in another embodiment, at least 55, or in another embodiment, at least 60, or in another embodiment, at least 65, or in another embodiment, at least 75 nucleotides in length, specifically hybridizable with the isolated nucleic acid of SEQ ID Nos: 1 or 2.

The oligonucleotide of this invention, according to another embodiment, may be in either sense or antisense orientation, may comprise DNA or RNA, and/or may be single or double stranded. The invention also provides, in other embodiments, compositions and/or vectors comprising the oligonucleotides of this invention.

In one embodiment, the oligonucleotide is in either sense or antisense orientation. In another embodiment, the oligonucleotide is either single or double-stranded. In another embodiment, the invention provides for a vector or composition comprising the oligonucleotides of the invention.

Techniques for introducing the above described recombinant nucleic acids and vectors, and others to be described hereinbelow, used in the present invention, are as described, and are to be considered embodiments of the invention for each instance of nucleic acid or vector delivery.

Determination of whether the methods employed disrupt TAK1-SMAD MH2 interaction may be accomplished through a variety of means well known in the art, including, but not limited to chemical cross-linking of the proteins, the yeast two hybrid system, and co-immunoprecipitation.

SMAD signaling pathways have been shown to be essential for bone formation, or osteogenesis. Osteoblast differentiation is SMAD-dependent, mediated via BMP-2 expression. Mesenchymal stem cells, in the presence of recombinantly expressed BMP2 undergo osteogenic differentiation via BMP-SMAD dependent signal transduction cascade.

In another embodiment, there is provided a method of stimulating or enhancing BMP-mediated SMAD activity comprising the steps of administering an agent that diminishes or abrogates TAK1 expression or function.

Mesenchymal stem cells engineered to constitutively express BMP2 demonstrate osteoblast differentiation, as compared to cells not expressing BMP2. Cells expressing BMP-2 yet further engineered to express TAK1, however, exhibited a phenotype consistent with mesenchymal stem cells, not expressing BMP2, i.e. TAK1 expression prevented osteogenic differentiation. Expression of the dominant negative TAK1 mutant demonstrated enhanced osteogenic differentiation.

As used herein the term “BMP” refers to the bone morphogenic family of proteins. BMPs bind their cognate receptors in order to exert their effect. Cells found to express both BMP type I and type II receptors include human mesenchymal stem cells (hMSCs).

Examples of members of the BMP family include, but are not limited to: BMP-1, BMP-2, BMP-3, BMP-4, BMP-5, BMP-6, BMP-7, BMP-8, BMP-9, BMP-11 and BMP-11.

The agent employed to diminish or abrogate TAK1 expression or function in BMP-mediated SMAD activity, or for any application in diminishing or abrogating TAK1 function is as defined above, and is to include, in one embodiment, physical inhibitors to TAK1-SMAD binding, as well as inhibitors of TAK1 expression, all of which are to be considered as part of the present invention.

The addition of BMP may, in one embodiment, precede or accompany the administration of the agent to diminish or abrogate TAK1 expression or function, as a means of enhancing or stimulating BMP-mediated SMAD activity, and represents another embodiment of the present invention.

In another embodiment, the nucleic acids, or vectors comprising an agent to diminish or abrogate TAK1 expression or function may further comprise a second nucleic acid that functions in osteogenesis.

The second nucleic acid may, in one embodiment, correspond to a nucleic acid, encoding the osteogenic factors OP-1, OP-2, BMP-5, BMP-6, BMP-2, BMP-3, BMP-4, BMP-9, DPP, Vg-1, 60A or Vgr-1, each of which represents an embodiment of the invention.

In another embodiment, there is provided a method of enhancing osteogenesis in a subject in need, comprising the steps of administering an agent that mitigates or abrogates TAK1 expression or function to a cell with osteogenic potential in the subject, thereby enhancing osteogenesis in said subject.

In other embodiments, the agent may be administered to the subject via direct delivery to a specific tissue site, such as, for example, by injection or catheterization, or indirectly via parenteral administration, by for example, osmotic pump delivery, aerosol exposure, and numerous methods well described in the art, each of which represents a separate embodiment of the present invention.

In another embodiment, administration can be accomplished in vitro, for example, by direct injection into a cell, or any other means well known to one skilled in the art. Indirect administration may be accomplished in vitro, for example, by supplementing a media surrounding a given cell with an agent, which enables the cell to ultimately be exposed to the agent. Ex-vivo culture of the cells is another embodiment of the invention, whereby a cell is contacted with an agent, a nucleic acid, a vector, a cell or a composition as described herein, then implanted into a subject in need.

In another embodiment, the cell may be loaded on a scaffolding material, prior to its administration to a host. By the term “scaffold” or “scaffolding material” it is meant, in one embodiment, to include a porous structural device that enables cell growth within the device, providing adequate nutrient exchange for cell growth. A scaffold can form a base which serves as a guide for tissue growth.

In one embodiment, the scaffold is formed of a bio-absorbable, or biodegradable, synthetic polymer such as a polyanhydride, polyorthoester, polylactic acid, polyglycolic acid, and copolymers or blends thereof. In another embodiment, non-degradable materials can also be used to form the scaffold. Examples of suitable materials include ethylene vinyl acetate, derivatives of polyvinyl alcohol, teflon, and nylon. In another embodiment, non-degradable materials are a polyvinyl alcohol sponge, or alkylation, and acylation derivatives thereof, including esters. A non-absorbable polyvinyl alcohol sponge is available commercially as Ivalon™, from Unipoint Industries. Methods for malting this material are described in U.S. Pat. No. 2,609,347 to Wilson; U.S. Pat. No. 2,653,917 to Hammon, U.S. Pat. No. 2,659,935 to Hammon, U.S. Pat. No. 2,664,366 to Wilson, U.S. Pat. No. 2,664,367 to Wilson, and U.S. Pat. No. 2,846,407. In another embodiment, non-biodegradable polymer materials can be used, including polymethacrylate and silicon polymers.

It is to be understood that the terms “administration” or “provision” or “contacting” are meant to be synonymous, and refer to both direct or indirect exposure as described herein, and that any mode of administration utilized for an agent, a nucleic acid, a vector, a cell or a composition as described herein, is to be considered within the framework of envisioned embodiments of this invention.

In, another embodiment of the invention, the agent mitigates or abrogates TAK1 expression or function following TAK1 activation by proinflammatory cytokines.

Inflammation is known to interfere with osteogenesis. As such, it may be desired to promote osteogenesis at a site of inflammation in a subject, including in instances where such inflammation is refractive to control measures, or the measures prove contraindicated for any number of reasons.

In another embodiment, inhibiting or abrogating TAK1 expression or function via the methods and agents described herein will be effective in the presence of any proinflammatory cytokine as well known in the art, including, for example, the pro-inflammatory cytokines IL-1 or TNF-alpha.

According to this aspect of the invention, osteogenesis is promoted via administration of the agent to a cell with osteogenic potential at an inflammatory site, or in another embodiment, via delivery of a cell with osteogenic potential to an inflammatory site.

As used herein the term “cell with osteogenic potential” refers, in one embodiment, to any cell that differentiates, or may be induced to differentiate to a cell that participates in bone deposition.

In another embodiment, a cell with osteogenic potential may be a mesenchymal stem cell, a progenitor cell, an osteoblast, or any cell capable of differentiating into an osteoblast.

In another embodiment of the invention, the subject in need suffers from inflammation-mediated bone loss, and thus stimulation of osteogenesis would serve a therapeutic purpose in the subject.

In another embodiment of the invention, the subject suffering from inflammation-mediated bone loss, may be treated locally at the site of inflammation or systemically. Systemic treatment with an agent downregulating or abrogating TAK1 expression or function may be accompanied by, in still other embodiments, the incorporation of additional moieties that help target the agent to the site of inflammation, including, but not limited to, the use of vector systems that additionally encode for targeting molecules including integrins, by methods well known in the art.

In another embodiment, the cell with osteogenic potential may be further engineered to express molecules that facilitate their targeting to specific sites of inflammation, following systemic delivery.

In another embodiment, the subject in need according to this aspect of the invention, suffers from periodontal disease, osteoarthritis, Köhler's bone disease, rheumatoid arthritis or osteoporosis. These diseases are not meant to be limiting however, and the methods, nucleic acids, vectors and compositions disclosed herein for promotion of osteogenesis may also find utility for subjects suffering from other diseases associated bone loss, including for example, some forms of osteomyelitis, and osteosarcoma. Each of these applications is to be considered an additional embodiment of the present invention.

In another embodiment of the invention, there is provided a method of enhancing osteogenesis in a subject in need, comprising the steps of genetically engineering a cell with osteogenic potential to be deficient in TAK1 expression or function and administering the engineered cell to the subject in need, thereby enhancing osteogenesis in the subject.

According to this aspect of the invention, in another embodiment, the cell may be further engineered to express a growth factor for stimulating or enhancing osteogenesis, such as for example a bone morphogenic protein, osteopontin or osteocalcin. In another embodiment, and as, disclosed above, the cell may be additionally engineered to express targeting moieties such as integrins. Anti-inflammatory cytokines may be expressed, inhibitors to enzymes associated with inflammation, such as inhibitor sequences to cyclooxygenase II, and other moieties that may diminish the inflammatory response. Each of these modifications represents an additional embodiment of the invention.

Osteogenesis is marked by the differentiation of progenitor osteoblasts, whose differentiation heralds their effector function in bone induction.

In one embodiment, osteogenesis stimulated or enhanced by the methods, agents, nucleic acids, vectors and/or compositions described herein may be measured via assessment of cell surface expression of osteopontin and BSP-II, which may be determined by FACS analysis, immuno-histochemistry or immunofluorescence assay. Osteogenesis, in other embodiments, may be determined via assaying cell alkaline phosphatase (ALP) activity by known histological techniques, or via assaying interleukin-6 (IL-6) and osteocalcin gene expression via methods well known in the art such as RT-PCR, Northern blot analysis or RNase protection assay of via assaying protein levels via Western blot analysis, ELISA or RIA.

Stimulation or enhancement of osteogenesis is, in one embodiment, beneficial not only in cases of bone loss, but in bone damage, as well. Such damage may be a result of any number of conditions, including, but not limited to bone fracture, genetic diseases, kidney disease, some infections or inflammation.

In another embodiment, there is provided a method of enhancing bone repair in a body of a subject in need comprising the steps of administering to a cell with osteogenic potential in said subject an agent that mitigates or abrogates TAK1 expression or function, thereby enhancing bone repair in a body of said subject.

In another embodiment, there is provided a method of enhancing bone repair in a subject in need, comprising the steps of genetically engineering a cell with osteogenic potential to be deficient in TAK1 expression or function, and administering, the engineered cell to the subject in need, thereby enhancing bone repair in the subject.

It is to be understood that the agent mitigating or abrogating TAK1 expression delivered to the subject, or the cell engineered to be deficient in TAK1 expression or function administered to the subject may comprise any of the embodiments listed above, and the agents, nucleic acids, vectors and compositions may comprise any of the embodiments listed herein, that result in the production of enhanced bone repair as a result of enhanced osteogenesis.

Changes in bone volume, quality or strength as a function of bone repair may be measured by a number of methodologies well known to one skilled in the art, including methods directly measuring tensile strength, and methods measuring various bone markers, as described in U.S. Pat. No. 5,785,041 by Weinstein et al., U.S. Pat. No. 5,509,042 by Mazess et al., Ronis M. J. J. et al. Toxicol Sci, (2001) 62: 321-329 or Suponitsky I. et al Journal of Endocrinology (1998) 156: 51-57. Bone repair may be taken as an improvement in measurements as above, as a function of the methods listed herein.

In addition to methods for enhancing BMP-mediated SMAD activity for stimulating osteogenesis, the invention also provides a means of diminishing or abrogating BMP-mediated SMAD activity thereby diminishing or abrogating osteogenesis.

In another embodiment, there is provided a method of diminishing or abrogating BMP-mediated SMAD activity, comprising administering an agent that stimulates or enhances TAK1 expression or function.

It is to be understood that the agent employed for any application herein of diminishing or abrogating BMP-mediated SMAD activity via stimulation or enhancement of TAK1 expression or function, is considered to encompass each embodiment described herein that provides for stimulated or enhanced TAK1 expression or function.

In another embodiment, there is provided a method of suppressing osteogenesis in a subject in need, comprising the steps of administering to a cell with osteogenic potential in said subject an agent that stimulates or enhances TAK1 expression or function, thereby suppressing osteogenesis in said subject.

As discussed, the agent employed for any application herein stimulating or enhancing TAK1 expression or function thereby suppressing osteogenesis, is considered to encompass each embodiment described herein, which provides for stimulated or enhanced TAK1 expression or function.

In another embodiment, any cell with osteogenic potential of the present invention may be administered to the subject at a site of lung injury or persistent infection.

Some diseases whereby local suppression of osteogenesis may be desired are diseases known to produce calcification of infectious foci, with accompanying expression of osteogenesis-associated genes, which result in the ultimate destruction of underlying tissue. Examples of such diseases may include histoplasmosis, tuberculosis and perhaps pulmonary alveolar microlithiasis (PAM), whose etiology is thought to be virally-mediated.

Other diseases whereby local suppression of osteogenesis may be desired include osteopetrosis and osteoma, and as such the use of the methods, agents, nucleic acids, vectors and compositions of the present invention may serve a therapeutic purpose for the subject. Each of these is to be considered additional embodiments of the invention.

A complete understanding of the complex molecular events leading to osteogenesis is currently lacking. In another embodiment of the invention, there is provided a method for the identification of candidate gene products involved in downstream events in BMP-mediated SMAD-signaling resulting in osteogenesis, comprising: (a) introducing an agent that inhibits or abrogates TAK1 binding to SMAD MH2 domains into a cell with osteogenic potential, (b) culturing a cell with osteogenic potential as in (a), without the agent, (c) harvesting RNA samples from cells in (a) and (b) separately, following stimulation of BMP-mediated SMAD-signaling, and (d) assessing differential gene expression, wherein differentially expressed genes in (a) as compared to (b) indicates that the gene is involved in downstream events in BMP-mediated SMAD-signaling resulting in osteogenesis.

In one embodiment, the cells are cultured both with and without the agent in vitro, and then RNA is harvested for differential expression analysis. In another embodiment, the cells are cultured ex-vivo both with and without the agent, then implanted into a subject, implanted cells are harvested at various times post implantation, and RNA harvested for differential expression analysis. In another embodiment, the agent is delivered to cells in vivo, cells are harvested at various times post agent delivery and RNA is harvested for differential expression analysis. In another embodiment, combinations of agent administration routes are conducted in parallel and differential gene expression is assayed concurrently among all experimental groups.

RNA may be extracted via a number of standard techniques (see Current Protocols in Molecular Biology” Volumes I-III Ausubel, R. M., ed. (1994); Ausubel et al., “Current Protocols in Molecular Biology”, John Wiley and Sons, Baltimore, Md. (1989)). Guanidium-based methods for cell lysis enabling RNA isolation, with subsequent cesium chloride step gradients for separation of the RNA from other cellular macromolecules, followed by RNA precipitation and resuspension, is an additional method of RNA isolation (Glisin, Ve. Et al (1973) Biochemistry 13: 2633). Alternatively, RNA may be isolated in a single step procedure (U.S. Pat. No. 4,843,155, and Puissant, C and Houdebine L M (1990) Biotechniques 8: 148-149). Single step procedures include the use of Guanidium isothiocyanate for RNA extraction, and subsequent phenol/chloroform/isoamyl alcohol extractions facilitating the separation of total RNA from other cellular proteins and DNA. Commercially available single-step formulations based on the above-cited principles may be employed, including, for example, the use of the TRIZOL reagent (Life Technologies, Gaithersburg, Md.

According to further features of this aspect of the present invention, monitoring differential gene expression may be accomplished via a number of standard techniques well described in the art, any of which can be employed to evaluate a given gene's expression. These assays comprise Northern blot and dot blot analysis, primer extension, RNase protection, RT-PCR, or in-situ hybridization. Differential expression of a known gene can be assessed using probes designed that are sequence specific. It is also possible to utilize chip hybridization and cluster analysis to determine multiply differentially expressed genes, including subtractive hybridization as a means of identifying differentially expressed sequences that represents genes involved in downstream events in BMP-mediated SMAD-signaling.

In another embodiment, this invention provides a method for the identification of an agent involved in stimulating or enhancing osteogenesis, comprising: (a) contacting a cell with osteogenic potential with an agent thought to inhibit or abrogate TAK1 interaction with SMAD MH2 domains; (b) culturing said cell with osteogenic potential under conditions facilitating TAK1-SMAD MH-2 interaction; and (c) determining whether said agent altered said TAK1-SMAD MH2 interaction, wherein altered TAK1-SMAD MH2 interaction as a result of contact with said agent produces stimulated or enhanced osteogenesis; thereby identifying an agent involved in stimulating or enhancing osteogenesis.

According to this aspect of the invention, in one embodiment, the agent may comprise any molecule that serves to inhibit or diminish SMAD MH2 interaction with TAK1, and is to be considered to comprise all embodiments listed herein.

In one embodiment, inhibited or diminished SMAD MH2 interaction with TAK1 may be measured by any number of methods well known to one skilled in the art, including methods exemplified herein, such as, in one embodiment, immunoprecipitation methods. Other methods include subcellular fractionation and subsequent immunoblot analysis, immuno-cytochemistry, and other methods herein described.

In another embodiment, inhibited or diminished SMAD MH2 interaction with TAK1 may be measured, via assessment of osteogenic differentiation in the cells. Inhibition of SMAD MH2 interaction with TAK1 results in stimulated or enhanced osteogenesis and may be evidenced via up-regulated or stimulated expression of osteogenic markers, as described herein. It is to be understood that any method that determines the presence of inhibited or abrogated TAK1-SMAD MH2 interaction, or enhanced osteogenesis via inhibited or abrogated TAK1-SMAD MH2 interaction, is to be considered as part of this invention, each of which represents an embodiment thereof.

The following examples further illustrate some aspects of the invention herein described. These examples, combined with the specification hereinabove, are intended is for exemplification purposes, and are not to be construed as a means of limiting the present invention.

### EXAMPLES

### Materials and Methods

Generation of TAK1-Expression Plasmids

Based upon the published sequence of murine TAK1 (Genbank Accession No. D76446) PCR primers were generated for cloning of wild-type (wt) TAK1 from murine kidney: forward 5′-TATAGAATTCCGCGGGGGATCATGTCGACAGCC (SEQ ID No: 5), and reverse 5′-TATAGGATCCTCATCACAGATCCTCTTCTG AGATGAGTTTTTGTTCTGAAGTGCCTTGTCGTTTCTGCTG (SEQ ID No: 6). The reverse primer additionally contained a sequence encoding a single c-myc-epitope sequence.

In addition, a constitutively active TAK1 (TAK1ca) lacking the 22 N-terminal amino, acids (Yamaguchi, K et al, Science, 270: 2008-2011, 1995) and a dominant-negative TAK1 (TAK1dn) mutant (K63W) (Genbank accession number XM_{—}131329), were generated with standard methods using a mutagenesis kit (Stratagene, LaJolla, CA). TAK1 variants were re-cloned into with C-terminal Flag- or HA-tags into pcDNA3. All TAK1 cDNA variants were confirmed by sequencing.

For verification of tissue-specific expression of murine TAK1 splice variants by PCR the following primers were used: forward: 5′-CAACTCAGCCACCAGCACAGG (SEQ ID No: 7), reverse: 5′-GACTGCGAGCTGGCTTCTCTG (SEQ ID NO: 8).

In addition, TAK1 deletion mutants were generated via PCR utilizing the following primers:

For the TAK1 Δ301-606 C-terminal deletion mutant (TAKDC):

TAK1wt-forward:

(SEQ ID No: 9)

TATAGAATTCGCCACCATGTCGACAGCCTCCGCCGCCTCG.
TAK1 Δ301-606-reverse

(SEQ ID No: 3)

TATAGGATCCTCATCACTTGTCATCGTCATCCTTGTAGTCATACTGTAAT
GGCTCATCCG:

For the TAK1 Δ1-300 N-terminal deletion mutant (TAKDN):

TAK1 Δ1-300 forward:

(SEQ ID No: 4)

TATAGAATTCGCCACCATGCCTTGTCAGTACTCTGATGA
TAK1wt-reverse

(SEQ ID No: 10)

TATAGGATCCTCATCACTTGTCATCGTCATCCTTGTAGTCTGAAGTGCCT
TGTCGTTTCTGCTG.

Cells and Transfection

Murine C3H10T½ progenitor cells were cultured in high-glucose DMEM containing 10% fetal calf serum, as described (Ahrens M et al., DNA Cell Biol, 12: 871-880, 1993). Cells were stably transfected with expression plasmids encoding wt TAK1, dominant-negative (dn) or constitutively active (ca) TAK1 in pMT7T3-f1 using DOSPER™ according to the manufacturer's protocol (Roche, Mannheim) and a selection plasmid conferring puromycin resistance (pBSpacDp: parental C3H10T½ cells) or G418-resistance (pAG60: BMP2-expressing C3H10T½ cells). Individual clones were picked, propagated, and tested for recombinant expression of TAK1-variants by RT-PCR using a vector-specific and a gene-specific primer. Control cell lines (empty expression vector) were established at the same time. Cells were plated at a density of 5,000 cells/cm². After reaching confluence (arbitrarily termed day 0) ascorbic acid (50 microgram/milliliter (μg/ml)) and b-glycerophosphate (10 millimolar (mM)) were added.

Histological Methods and Verification of Cellular Phenotypes

Osteoblasts exhibit a stellate morphology and display high levels of alkaline phosphatase activity, which was visualized by cellular staining with SIGMA FAST BCIP/NBT (Sigma, Deisenhofen). Proteoglycan-secreting chondrocytes were identified by staining with Alcian Blue at pH 2.5 (Alcian Blue 8GS, Roth, Karlsruhe).

RNA Preparation and Semi-Quantitative RT-PCR

Total cellular RNA was prepared by TriReagent LS according to the manufacturer's protocol (Molecular Research Center Inc., Ohio, MS). Five μg of total RNA was reverse transcribed with oligo-dT primers and cDNA aliquots were subjected to PCR. RT-PCR was normalized by the transcriptional levels of HPRT. The primer pairs and PCR conditions used to evaluate osteo-/chondrogenic differentiation, for collagen Ia1, collagen IIa1, osteocalcin, and the PTH/PTHrP-receptor have been described (Hoffmann A et al, J. Cell Sci 115: 769-781, 2002).

Western Blotting

Western Blotting was performed as described (Hoffmann A et al, ibid). In short: Recombinant cells cultured in Roux flasks were harvested at different time points in TriReagent LS. Protein was isolated, dissolved in SDS containing sample buffer and equal amounts (total protein) were subjected to polyacrylamide gel electrophoresis. Proteins were transferred to nitrocellulose membranes. After blocking, membranes were incubated with a rabbit antibody to TAK1 (SC-805, Santa Cruz. Biotechnology, Santa Cruz Calif.) diluted 1:1,000 in, blocking solution. The secondary antibody, horseradish-peroxidase-conjugated goat anti rabbit IgG (H+L, Dianova, Hamburg), was applied at 1:10,000 in blocking solution for 1 hour at room temperature and detected by chemoluminescence (ECL, AP Biotech, Freiburg).

Co-Immunoprecipitation from HEK293T Cells

Co-immunoprecipitations were conducted as described (Verschueren K et al, J. Biol. Chem, 274: 20489-20498, 1999). Human embryonic kidney (HEK) 293T cells were plated at 3×10⁶ cells/petri dish (55 square centimeters (cm²)) one day prior to transfection. For transient transfections, 1 μg of Flag-tagged expression plasmid and 2 μg of Myc- or HA-tagged expression plasmids were utilized. DNA content was normalized with empty vector. Transfections were performed with FuGENE6 (Roche, Mannheim). Roughly 36 hours post-transfection cells were dislodged from dishes and harvested by centrifugation. Pellets were shock frozen in liquid nitrogen. IP reactions were performed in lysis buffer. Cell extracts and immunoprecipitates were analyzed by SDS-PAGE and Western blot analysis using the appropriate antibodies.

Reporter Assays

Reporter assays using the GAL4 DNA binding domain fused to various forms of SMAD proteins were performed as described (Meersseman, G et al,. Mech. Dev, 61: 127-140, 1997). Briefly, 420 nanograms (ng) DNA was transfected/well (24 well-plate seeded with 1×10⁵ HEK 293T cells) one day prior to transfection. The DNA mix included a plasmid expressing β-galactosidase under the control of the RSV promoter, for normalization purposes and a reporter plasmid encoding firefly luciferase under the control of 5 GAL4 binding sites upstream of a minimal TATA box (pG5 luc, Promega, Mannheim) and combinations of effector plasmids. The DNA content was normalized with empty vector.

Cells were harvested and lysed. β-Galactosidase (β-gal) activity was measured via the luminescent β-galactosidase kit II (Clontech, Palo Alto, Calif.), and luciferase activity via the luciferase assay system (Promega, Mannheim). All results are expressed as luciferase activity normalized with β-gal values (“relative luciferase activities”). Reporter assays with the SMAD1 DNA binding element SBE-9 (GCCG×9) (Kusanagi K et al, Mol Biol Cell: 555-565, 2000) cloned into pCAT5 vector (Boshart M et al, Gene 110: 129-130, 1992) were performed similarly except that cells were lysed in CAT lysis buffer. CAT enzyme was measured via CAT ELISA assay and β-gal via chemoluminescent β-gal reporter gene assay (Roche, Mannheim).

Immunofluorescence

HEK293T cells were seeded at 2×10⁵ cells/well into poly-D-lysine coated 6-well plates one day prior to transfection. Transient transfections were conducted with Fugene 6. After 30 hours, the cells were fixed with 4% paraformaldehyde in phosphate buffered saline (PBS) for 10 minutes, washed twice with PBS, and permeabilized with methanol followed by 0.5% Triton-X 100 in PBS. After washing with 0.1% Triton-X 100 in PBS (PBT), blocking was performed with 5% FCS in PBT for 1 hour at room temperature. Following another PBT wash, tagged proteins were incubated with the appropriate primary antibodies. Detection was accomplished via goat anti mouse-Alexa 488 (A-11001, Molecular Probes) (5 μg/ml) antibody for murine primary antibodies.

Flourescence Activated Cell Sorting for DNA Content/Cell Cycle Distribution

Exponentially growing cells were harvested 48 hours post-seeding or at different stages at and after confluence by trypsinization and fixation with 80% methanol. Cellular DNA content was determined by staining cells with RNAseS/propidium iodide, and measuring fluorescence in a Becton Dickinson FACScan. For each cell population, 10,000 cells were analyzed, and the proportions in G₀/G₁, G₂/M and S-phases were estimated by using Modfit cell cycle analysis software. Information on cell size (e.g. due to differentiation) and complexity could be obtained from forward and side scatter data following evaluation of relevant cell populations.

In Vivo Transplantation of C3H10T½ Cells Co-Expressing TAK1 dn and BMP2

1×10⁶ C3H10T 1/2 cells co-expressing TAK1dn and BMP2 are loaded onto a collagen carrier scaffold (3×3×3 mm). TAK1 wildtype C3H10T½ cells co-expressing BMP2 are similarly prepared. Loaded scaffolds are transplanted ectopically into the abdominal muscle of C3H/HeN female mice (8 weeks old). Alternatively 5×10⁶ C3H10T½ cells co-expressing BMP2 and TAK1dn or TAK1 wildtype cells are injected into the abdominal muscle, without any scaffolding.

At two and four weeks post-transplant/implantation, mice are sacrificed, and transplants/implanted cells are harvested and analyzed for bone formation.

Analysis of In Vivo Bone Formation

Samples are evaluated by micro CT for determination of the amount of bone formed. Following imaging analysis, samples are processed for H&E, Masson Trichrome and alcian blue staining, for histological evaluation, for further evaluation of bone formation.

### Example 1

### Biologically Active TAK1 Splice Variants

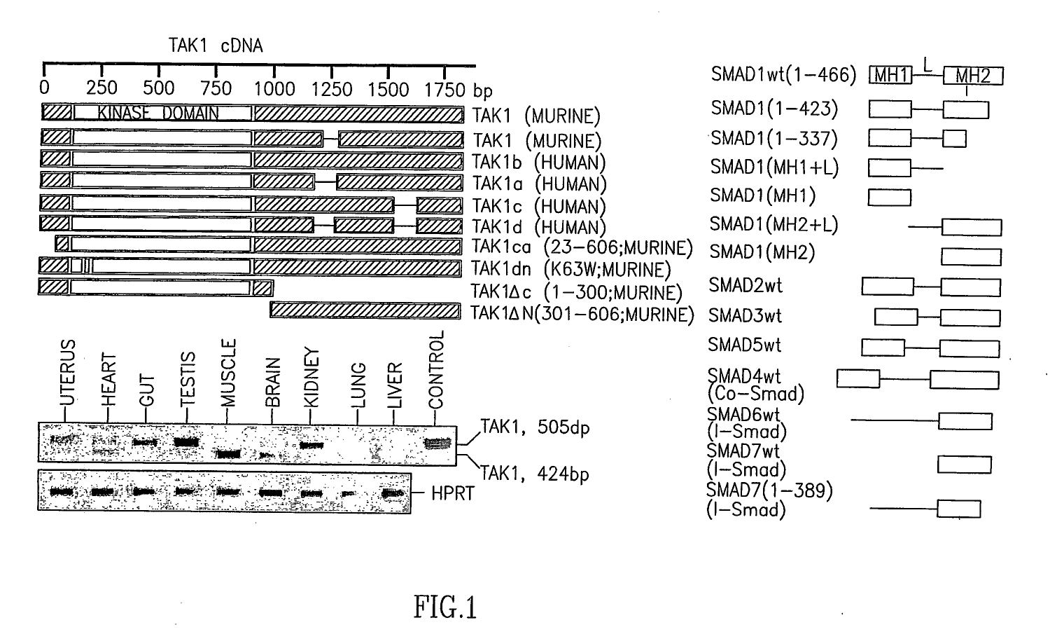

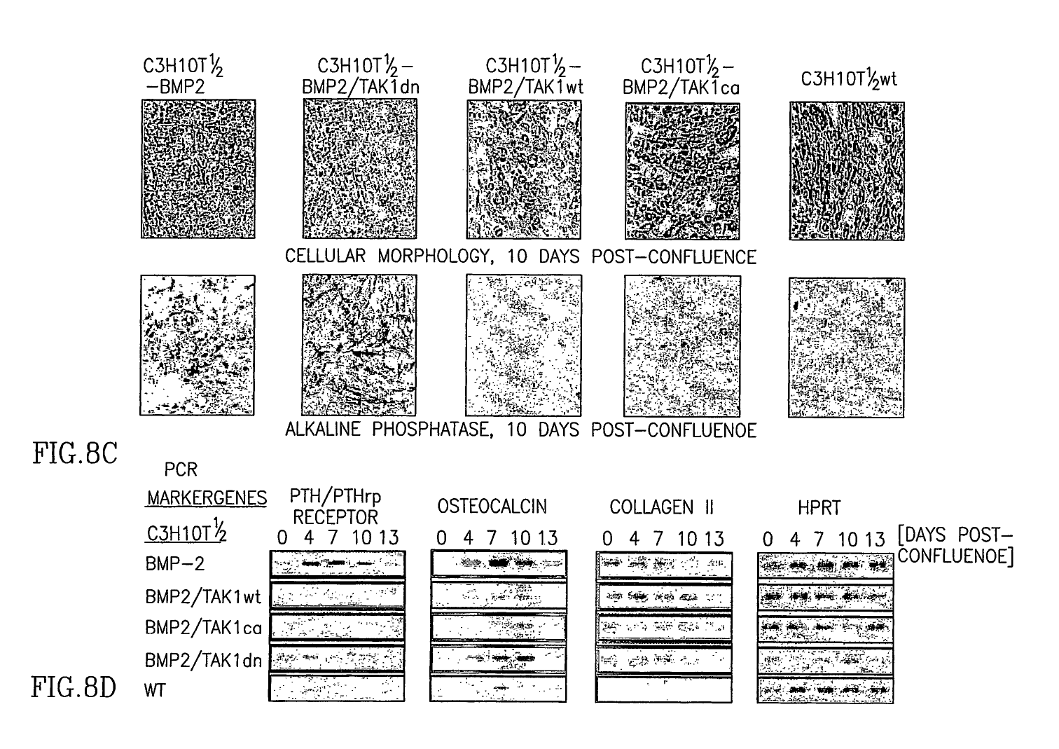

To determine whether TAK1 expression and biological activity were comparable in tissue and in C3H10T½ cells, full-length TAK1 cDNA was generated by PCR from total murine kidney RNA and in the mesenchymal progenitor cell line C3H10T½. The isolated TAK1 cDNA exhibited a high degree of homology to human TAK1b, which has since been confirmed (Genbank accession number XM_{—}131329; FIG. 1A), and is a longer transcript form than one previously described (Yamaguchi K et al, 1995, ibid). Among nine tissues examined, both TAK1 long and short forms were expressed in every tissue assessed apart from kidney (FIG. 1B). TAK1 tissue-specific expression levels varied, with minimal lung expression detected. Both TAK1 splice variants were comparably expressed in the mesenchymal progenitor line, C3H10T½(see FIG. 8A). TAK1 mutants generated from the long transcript form included dominant-negative (TAK1dn) and constitutively active (TAK1ca) variants as compared to wild-type control (TAK1wt). TAK1wt and TAK1ca were biologically active, as evidenced by p38 MAPK and JNK phosphorylation as a result of their expression in transfected cells (data not shown).

### Example 2

### TAK1 Preferential Interaction with Latent SMADs

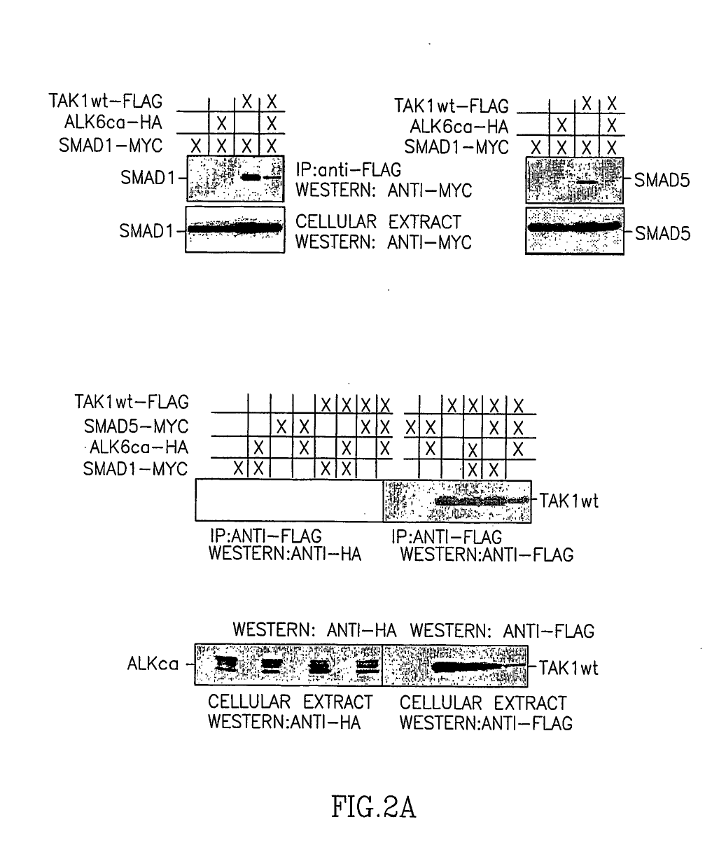

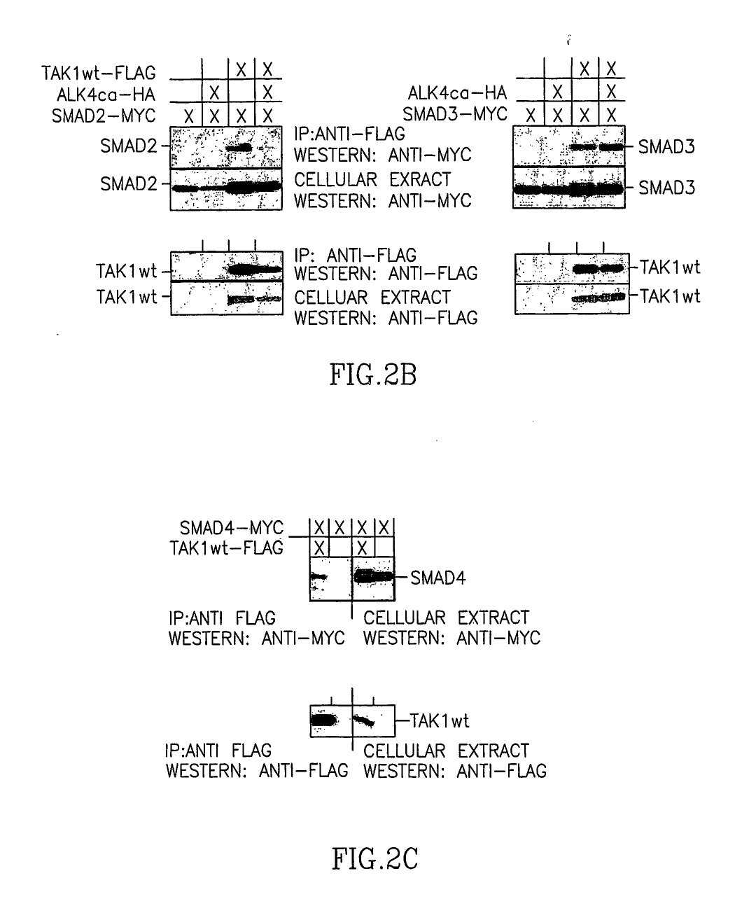

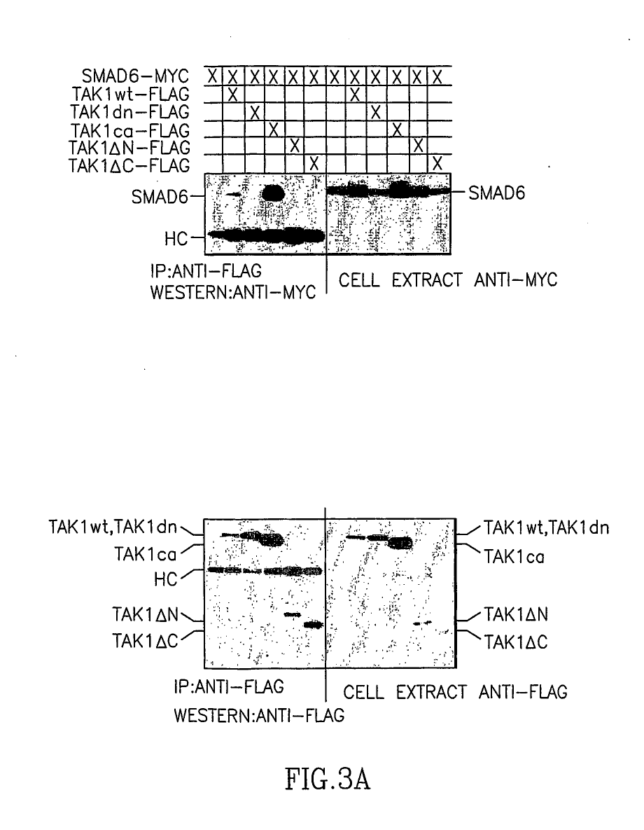

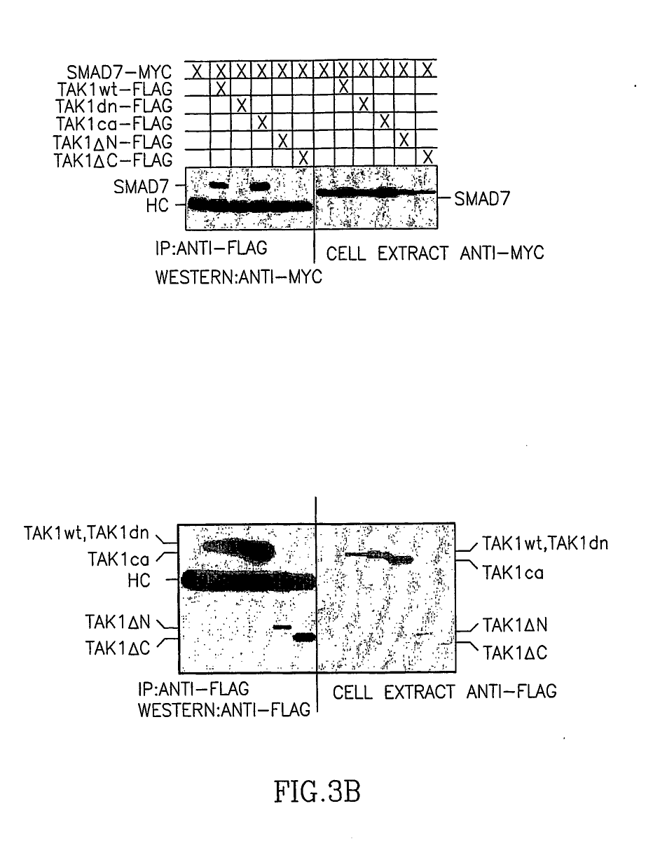

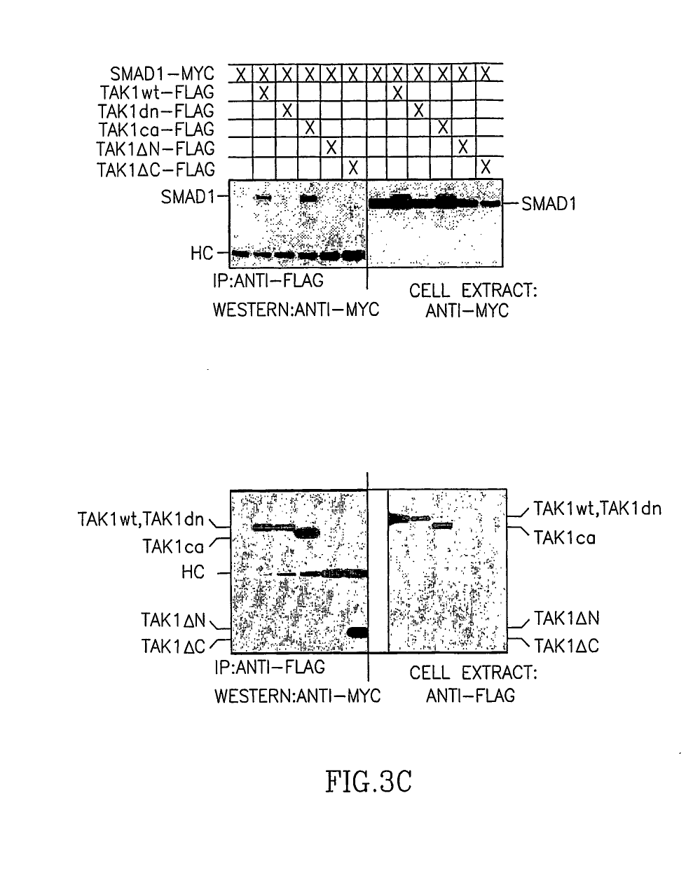

Since TAK1 is activated by ligands of the TGF-β and BMP family, and SMADs are involved in signaling cascades of the latter, it was important to determine whether direct SMAD interaction with TAK1 in cells can be demonstrated. Toward this end, co-immunoprecipitations with various combinations of wild-type TAK1 (TAK1wt) and R-SMADs, either in a latent form or post-activation by constitutively active BMP type I receptor (ALK6ca, BMPR-IB) or activin type I receptor ALK4ca (ActR-IB), I-SMADs and SMAD4, were conducted. TAK1 was found to co-immunoprecipitate, hence interact with, all R-SMADs tested (FIG. 2). TAK1 interaction with R-SMADs was typically stronger in the absence of constitutively active receptors (the exception being SMAD3), which may reflect a reduced affinity for activated, i.e. phosphorylated R-SMADs by TAK1, except with SMAD3. Other members of the SMAD family of proteins also exhibited a significant affinity for TAK1. SMAD4 and the I-SMADs, SMAD6 and SMAD7 efficiently bound to TAK1 (FIG. 2C; FIGS. 3A,B), confirming previous reports for SMAD6 (Kimura N et al., J Biol Chem, 275: 17647-17652, 2000).

One explanation for TAK1 preferential interaction with latent SMADs is TAK1-mediated competition with ligand-activated receptors for the availability of latent cytosolic SMADs.

SMAD proteins only bind TAK1 with an intact or a constitutively active kinase domain. Different TAK1 variants such as TAK1wt, TAK1ca, TAK1dn and two deletions, TAKDN (lacking amino acids 1-300; FIG. 1a) and TAKDC (lacking 301-606; FIG. 1a), respectively, were tested for their ability to bind to R-SMADs (using SMADL as a representative) or I-SMAD6 and I-SMAD7. Only the kinase active form of TAK1 was able to, bind efficiently to SMAD proteins as the point mutation K63W, which interfered with the kinase activity of TAK1, blocked binding of TAK1 to SMADs. There were minor differences between the two I-SMADs: SMAD6 had lower affinity for TAK1 than SMAD7 since it efficiently bound to TAK1ca only. In contrast, SMAD7 and the R-SMADs 1-3, 5, and SMAD4 were able to interact with both TAK1wt and TAK1ca (FIG. 2, FIG. 3). Although TAK1ca exhibited a slight binding capacity for SMAD1, the kinase-containing C-terminal domain of TAK1 was necessary for SMAD interaction. The two TAK1 deletion mutants TAKDN and TAKDC were unable to bind significantly to SMADs (FIG. 1a, FIG. 3).

### Example 3

### SMAD MH2 Domains Mediate TAK1 Binding

SMAD deletion mutants were prepared in order to determine which domains were necessary and sufficient for TAK1 binding. Since all SMADs were found to bind TAK1 (FIG. 1 c) and the MH2 domain is the only structural motif common to all it seemed the likeliest candidate for TAK1 binding. Indeed, for both SMAD1 and SMAD3 the MH2 domain sufficed to mediate TAK1 interaction, with MH1 and linker domains not involved in the TAK1 interaction (FIG. 3a, b).

The MH2 C-terminal sequences of R- , Co- and I-SMADs demonstrated the greatest degree of homology among SMADs, with the exception of a few terminal amino acids. The terminal amino acids are subjected to receptor-mediated phosphorylation in R-SMADs and thus differ from co-SMAD4, and are absent in I-SMADs.

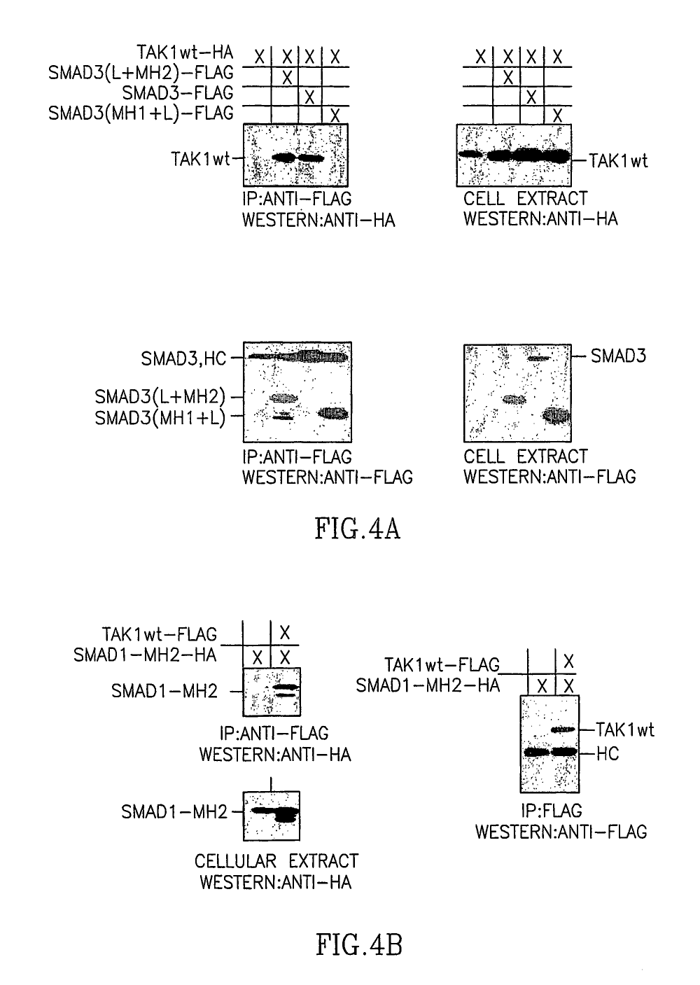

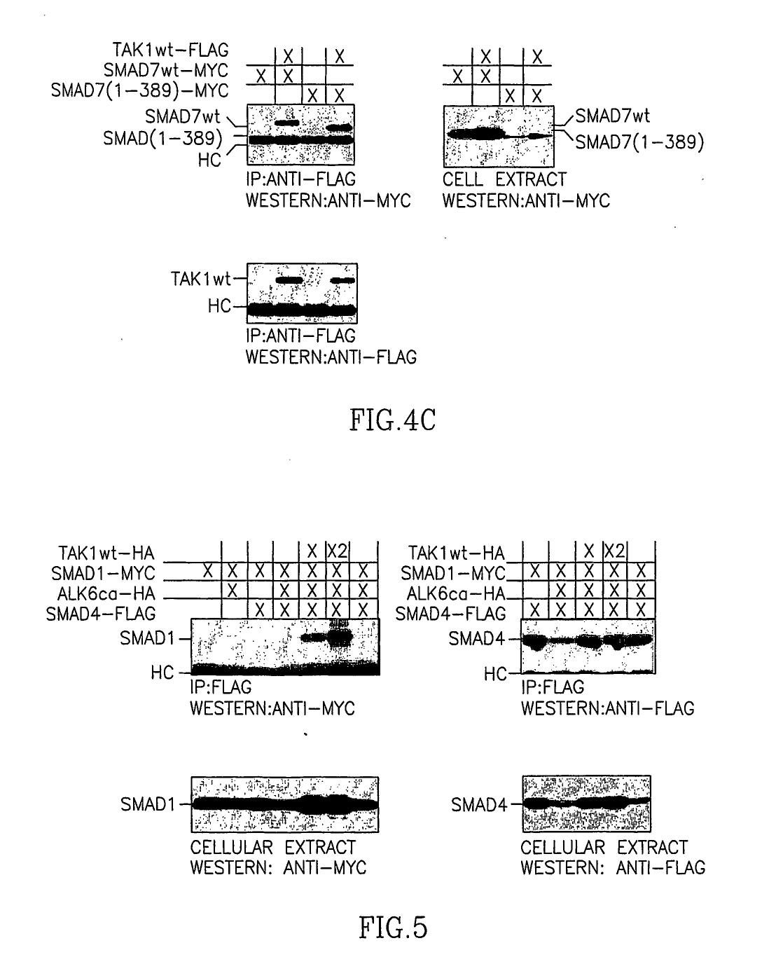

MH2 C-terminal sequences do not mediate TAK1 binding, despite their highly conserved nature. SMAD7 deletion mutants (1-389) lacking 38 C-terminal conserved amino acids common to all SMAD family members retained full binding ability compared to fall-length SMAD7, indicating that the TAK1 binding is located upstream of the distal end of this MH2 domain (FIG. 4c).

### Example 4

### TAK1 Binding to and Interference with SMAD Activity is a Mediated via Discrete SMAD MH2 Domains

R-SMAD hetero-oligomerization with SMAD4 and subsequent accumulation in the nucleus is essential for biological activity. Weak receptor-mediated hetero-oligomerization of SMAD1:SMAD4 (FIG. 5) was greatly enhanced in the presence of TAK1.

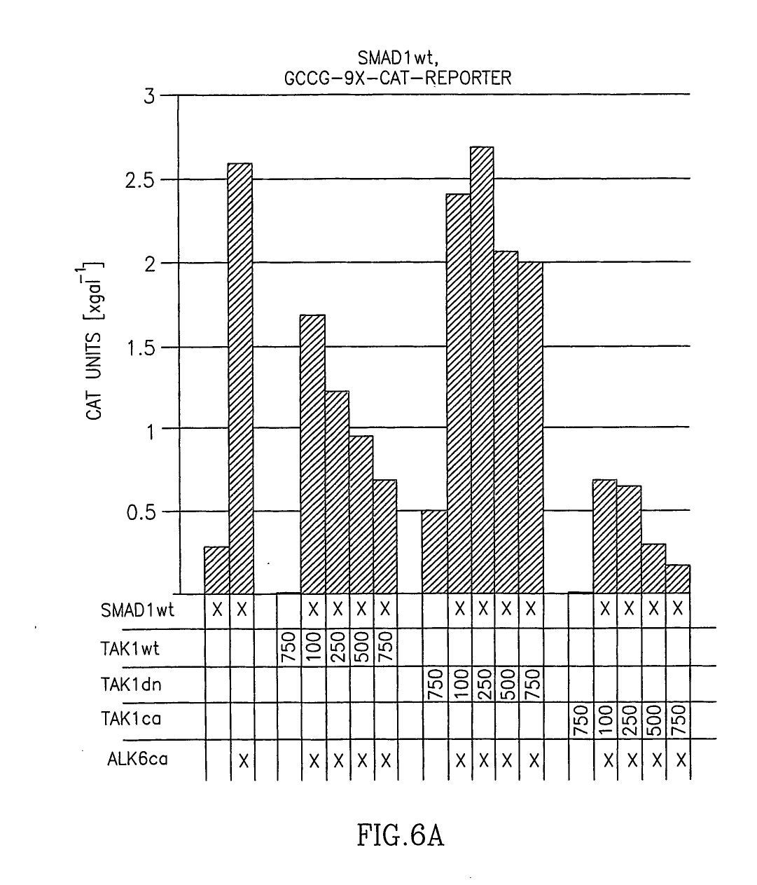

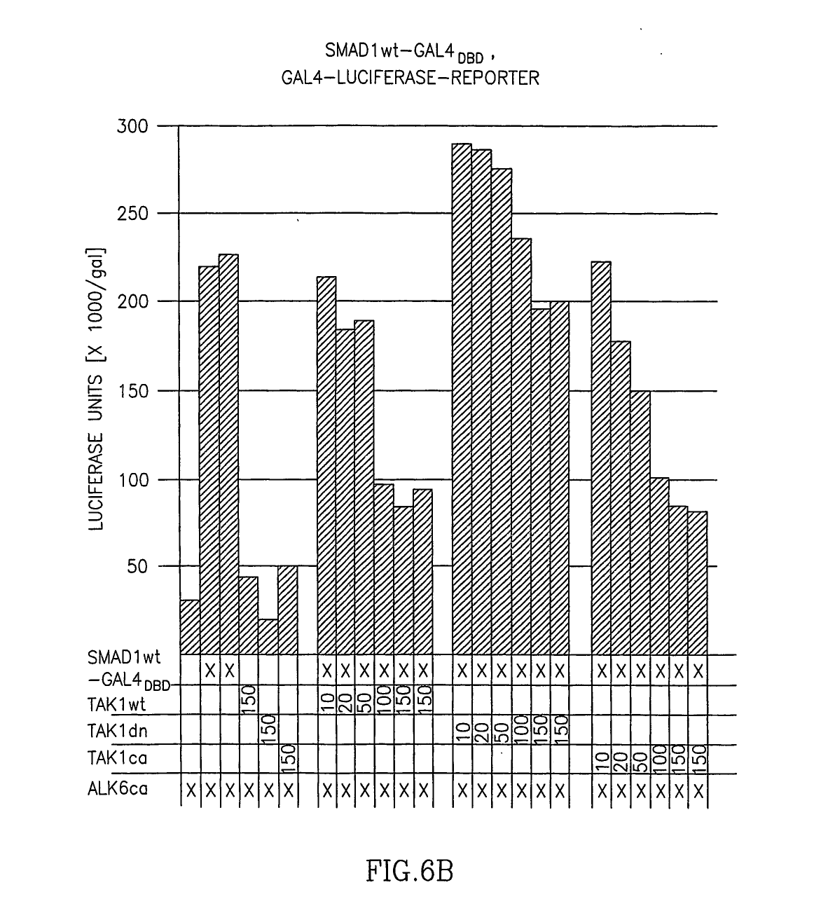

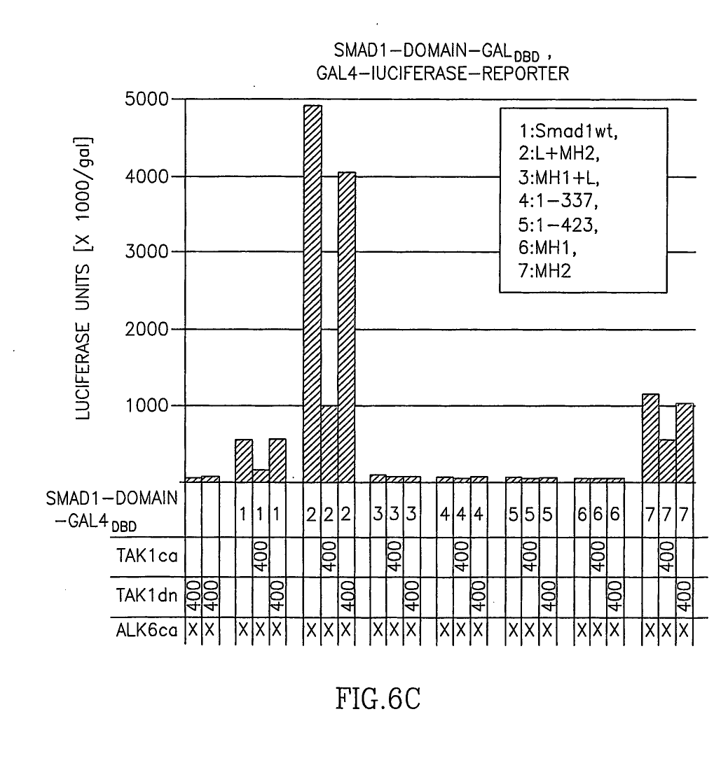

In order to determine whether TAK1-enhanced, receptor-mediated hetero-oligomerization of R-SMAD with SMAD4 had any effect on R-SMAD biological activity, a reporter assay measuring R-SMAD transcription was conducted. The SMAD1-SBE (Kusanagi K et al, Mol Biol Cell, 11: 555-565, 2000) was fused to a minimal promoter directing CAT reporter synthesis and transcription of SMAD1 in the presence of TAK1 was determined as a function of CAT expression (FIG. 6a). The addition of wild-type TAK1 and TAK1ca (30% and 10% maximum reduction, respectively) interfered with SMAD 1 transcription in a dose-dependent manner, following activation by constitutively active receptors. TAK1dn, however, demonstrated minimal inhibition.

To determine whether MH2 domains involved in TAK1 binding mediated inhibition of R-SMAD biological activity, a reporter assay measuring SMAD trans-activation potential was performed. Full-length SMAD1 and deletion mutants thereof were fused in-frame with the GAL4-DNA binding domain, and SMAD trans-activation potential was measured via UAS-driven-luciferase reporter expression (FIGS. 6B & C). The trans-activating potential of SMAD1/GAL4 hybrid molecules was measured with increasing amounts of TAK1 variants. Wild-type TAK1 and TAK1ca inhibited caALK6-receptor-mediated trans-activation of SMAD1 in a dose-dependent manner (FIG. 6B) (to a maximum of 40 with less TAK1 ca required for inhibition as compared to wild-type). In contrast, SMAD trans-activation increased by 35%, with the addition of low concentrations of TAK1dn, which may have functioned to compete with endogenous TAK1-like proteins in the host cell. The sharp drop in trans-activation activity from this elevated value to roughly 70% may be due to residual TAK1 activity of the dominant negative TAK1 (K63W) mutation (FIG. 6B).

Constructs encoding GAL4 fused to the SMAD1-MH2 or the SMAD1-L+MH2 domain demonstrated a significantly increased basal level of transcriptional activity in contrast to the fusion protein containing full-length SMAD1. Interestingly, the (L+MH2)-construct was much more efficient than the MH2-construct itself due to the presence of the proline-rich linker domain. The trans-activating potential of both constructs was significantly decreased in the presence of TAK1ca whereas TAK1dn had no effect (FIG. 6C).

Thus active TAK1 negatively interfered with SMAD1-dependent transcriptional activation, with the SMAD MH2 C-terminal end dispensable for TAK1 is binding (FIG. 4C), yet required for R-SMAD trans-activation.

### Example 5

### TAK1 Interferes with Nucleocytoplasmic SMAD Shuttling

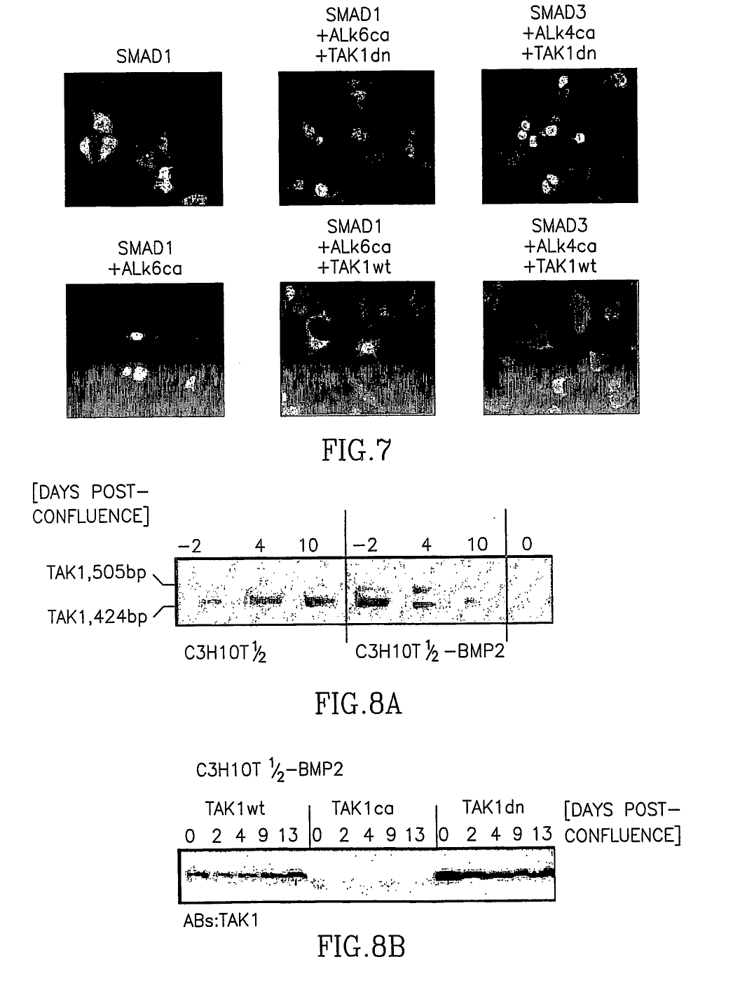

To determine whether TAK1-dependent interference with SMAD activity affected subcellular distribution of SMAD, immunofluorescence was used to localize flag-tagged SMAD1 or SMAD3 expressed in HEK293T cells in the absence or presence of constitutively active receptors (FIG. 7).

Uniform subcellular distribution of SMAD1 and SMAD3 was evident, in the absence of constitutively active receptors, while the SMADs accumulated predominantly in the nucleus in stimulated cells (FIG. 7). SMAD1 and SMAD3 accumulated active TAK1 (TAK1wt), and predominantly in the nucleus in the presence of the dominant-negative form of TAK1 (TAK1dn). Thus TAK1 activity or signaling affected nucleo-cytoplasmic shuttling of SMAD family members.

### Example 6

### TAK1 Activity Interferes with BMP-Dependent Osteogenic Differentiation of Mesenchymal Stem Cells

Osteoblast differentiation is SMAD-dependent, mediated via BMP-2 expression. The murine mesenchymal stem cell line C3H10T1½ cells, in the presence of recombinantly expressed BMP2 undergoes osteo-/chondrogenic differentiation. In this cellular system, osteogenic differentiation but not chondrogenic differentiation is predominantly mediated by BMP SMADs.

To determine TAK1 effects on SMAD-dependent osteogenesis, TAK1 expression was analyzed in C3H10T½cells, wild type cells or cells engineered to constitutively express BMP2. Expression of mRNA splice variants steady-state levels of the two did not change significantly during two weeks of culture, whether or not BMP2 was concurrently expressed (FIG. 8A). Individual stable cell lines were established expressing recombinant TAK1wt, TAK1ca or TAK1dn both in wild type and BMP2 expressing cells. TAK1 protein production was verified by Western blot analysis probing with anti-TAK antibodies specifically recognizing recombinant, and not endogenous TAK1 (FIG. 8B). TAK1ca expression in C3H10T½-BMP2 was considerably lower than of TAK1wt or TAK1dn, indicative of a cytotoxic TAK1ca effect.

Osteoblast formation was evaluated as a measure of TAK1 influence on osteo-/chondrogenic differentiation potential. C3H10T½-BMP2 cells co-expressing TAK1 and its molecular variants were assessed histologically for alkaline phosphatase (ALP) activity (FIG. 8C), which is indicative of osteoblast differentiation.

Unlike WT cells, which grow in a monolayer, and exhibit typical fibroblast morphology (right panel), C3H10T½-BMP2 cells grow in multiple layers once confluent (left panel), indicative of their differentiation along the osteo-/chondrogenic pathway. Multiple layer growth occurred in cells expressing TAK1dn, as well. Cells over-expressing TAK1wt or TAK1ca in C3H10T½-BMP2, however, demonstrated a C3H10T 1/2 wild-type phenotype.

Only control C3H10T½-BMP2 and TAK1dn over-expressing C3H10T½-BMP2 cells were ALP-positive (i.e. osteoblast-like) though. Surprisingly, C3H10T½-BMP2/TAK1dn cells exhibited enhanced ALP activity, as compared to C3H10T½-BMP2 cells, indicating that activated endogenous TAK1 negatively impacts osteogenic differentiation.

Osteogenic marker gene expression as determined by RT-PCR is enhanced in C3H10T½-BMP2 cells, as compared to wt cells, yet TAK1 co-expression down-regulates this response (FIG. 8D). C3H10T½-BMP2 cells demonstrated strongly up-regulated expression of osteocalcin and the parathyroid hormone receptor (PTH/PTHrP-receptor), as compared to parental mock-transfected controls. Co-expression of either TAK1wt or TAK1ca resulted in down-regulated expression of osteogenic genes. TAK1dn co-expression, however, had no effect (FIG. 8D). Thus both histological analysis and marker gene expression indicated that osteogenesis is negatively regulated by biologically active TAK1, and that BMP2-mediated activities seem to be specifically blocked because BMP2 expressing cells resume wild-type phenotypes in the presence of the introduced TAK1wt or TAK1ca.

In contrast to a marked TAK1-mediated effect on osteogenesis, however, chondrogenesis was unaffected. Collagen type II, an early marker of chondrogenic differentiation, was expressed at low levels in C3H10T½wt cells, whereas in C3H10T½-BMP2 cells, collagen type II mRNA expression was up-regulated (FIG. 8D). Steady-state mRNA expression levels did not change, regardless of TAK variant co-expression. No differences in chondrocyte content were detected by preferential staining of acid proteoglycans in chondrocyte extracellular matrix with Alcian Blue (data not shown), regardless of TAK variant co-expression; hence chondrocyte formation was not affected by TAK1 expression.

### Example 7

### Interference by TAK1 with Osteogenic Development is not Due to Induction of Apoptosis

To determine whether TAK1-mediated effects in the assay systems above resulted from apoptosis, C3H10T½-BMP2 cells co-expressing TAK1 variants were assayed by FACS for apoptosis and cell cycle progression.

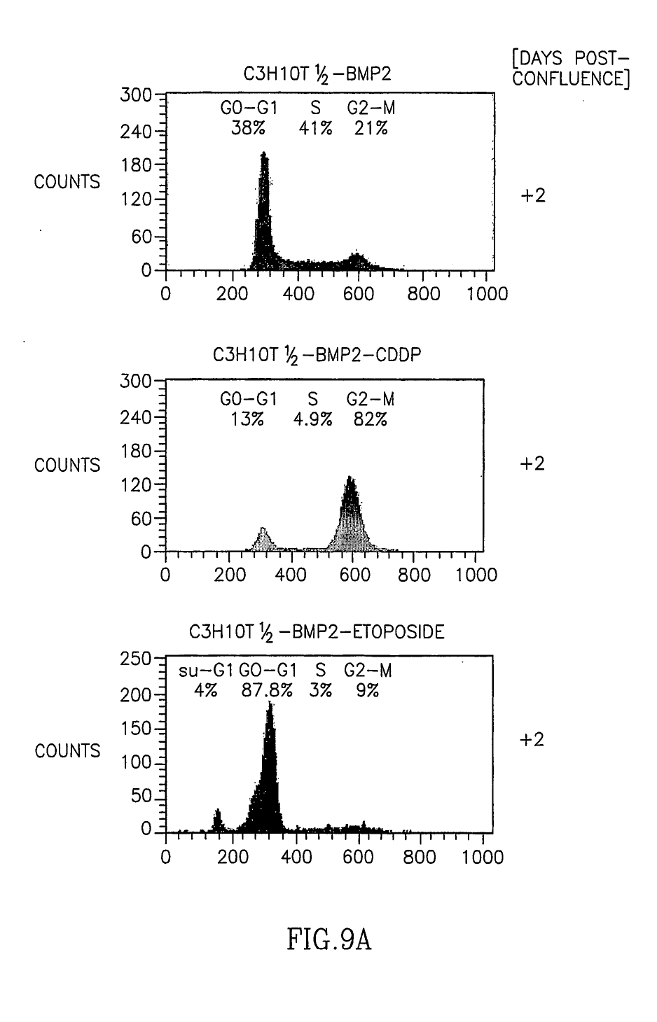

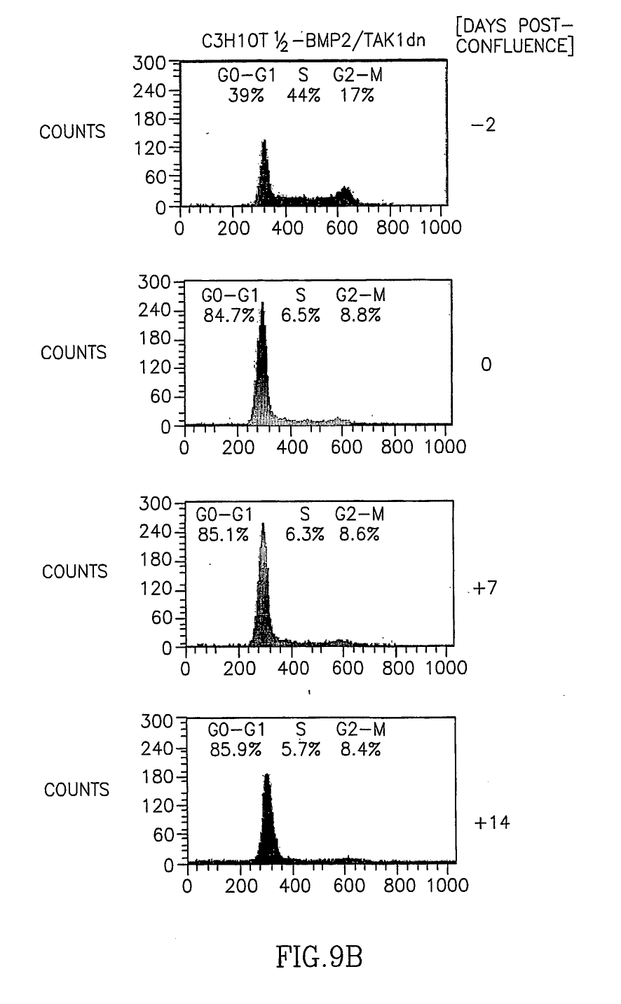

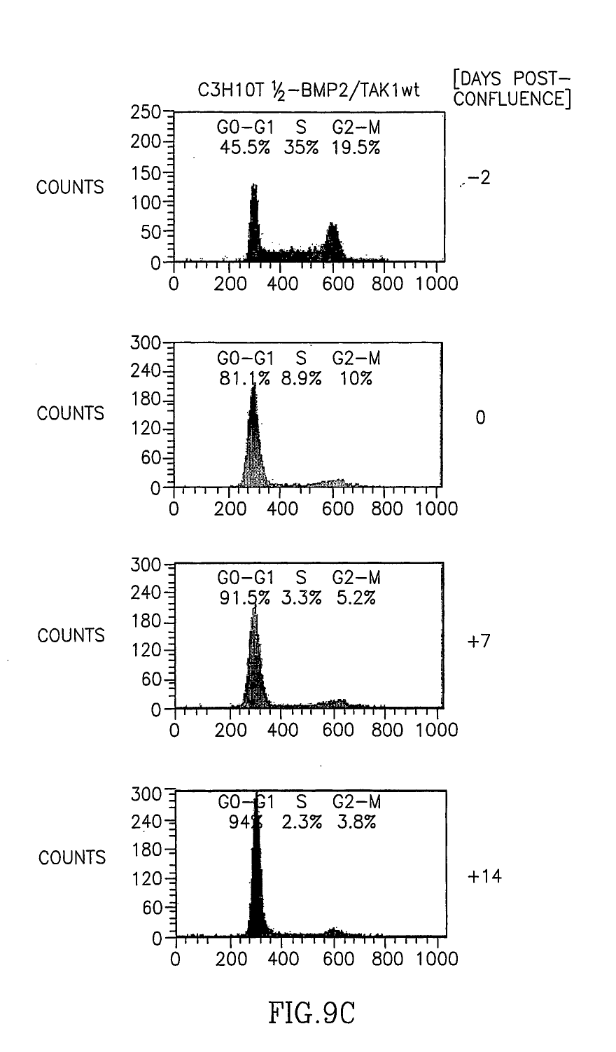

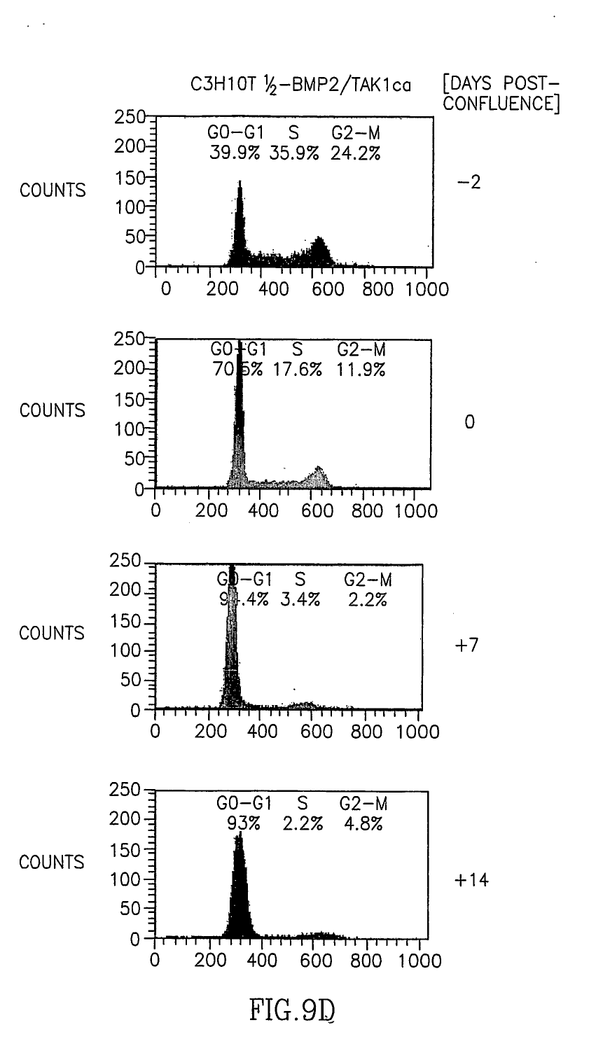

C3H10T½-BMP2 cells were treated with cisplatin (CDDP) or etoposide, agents known to induce apoptosis (FIG. 9). CDDP did not cause measurable apoptosis (though cell size was markedly increased; data not shown), but rather arrested maturation in the G2-M phase of the cell cycle (FIG. 9A). In contrast, etoposide induced a significant subG0-G1 peak and a shoulder in the G0-G1 peak accompanied by morphological changes (rounded cells) indicating ongoing apoptosis (arrows, FIG. 9A).

TAK1 variant co-expression in C3H10T½-BMP2 cells did not induce apoptosis. Regardless of the TAK1 variant expressed, cell cycle progression was similar, during cellular exponential growth phase (prior to confluence), at confluence, and thereafter (FIG. 9B-D). At confluence, the number of cells in S-phase or G2/M-phase was relatively negligible. At no time point analyzed were TAK1 expressing cells in sub-G1, hence no apoptosis was detected.

### Example 8

### In Vivo Bone Formation in Cells Co-Expressing TAK1dn & BMP2

C3H10T ½-BMP2 cells co-expressing TAK1dn demonstrated osteoblast differentiation in vitro. It is therefore of interest to determine whether these cells are capable of stimulating bone formation in vivo.

Toward this end, C3H10T½-BMP2 cells co-expressing TAK1dn are loaded on scaffolding material, and then transplanted into recipient mice, and in vivo bone formation is evaluated. Similarly, C3H10T/½-BMP2 cells co-expressing TAK1dn are implanted in mice. Controls include cells similarly prepared, but expressing the TAK1 wildtype sequence. Bone formation is assessed in each case, and compared among the groups, in order to determine the extent of mesenchymal stem cell osteogenic differentiation and its effect on bone formation, in cells where TAK1 interaction with BMP2-SMAD signaling is disrupted.

1-86. (canceled)

87. A method of regulating an activity of a SMAD protein in a cell, the method comprising contacting the cell with an agent capable of modulating an expression and/or an activity of TAK1 in the cell, thereby regulating the activity of the SMAD protein in the cell.

88. The method of claim 87, wherein said regulating the activity of the SMAD protein is stimulating or enhancing the activity of the SMAD protein, and whereas said modulating said expression and/or said activity of TAK1 is diminishing or abrogating said expression and/or said activity of TAK1.

89. The method of claim 87, wherein said agent comprises a polypeptide encoded by a nucleic acid having a nucleotide sequence at least 70% homologous to SEQ ID NO: 1 and/or SEQ ID NO: 2.

90. The method of claim 87, wherein said agent comprises a single-stranded or double-stranded oligonucleotide which is at least 12 nucleotides in length and is specifically hybridizable with SEQ ID NO: 1 and/or 2.

91. The method of claim 87, wherein said agent comprises an oligonucleotide having a nucleic acid sequence at least 70% homologous to SEQ ID NO: 3 and/or 4.

92. The method of claim 87 wherein said activity of TAK1 is a kinase activity and/or an interaction of TAK1 with an MH2 domain of the SMAD protein.

93. The method of claim 87, wherein said regulating the activity of the SMAD protein is diminishing or abrogating the activity of the SMAD protein, and whereas said modulating said expression and/or said activity of TAK1 is stimulating or enhancing said expression and/or said activity of TAK1.

94. A method of regulating osteogenesis and/or bone repair in a subject in need thereof, the method comprising contacting a cell with osteogenic potential with an agent capable of modulating an expression and/or an activity of TAK1 in the cell, wherein:
(i) said cell is located in the subject; and/or
(ii) said contacting is effected in-vitro, thereby generating a treated cell, and the method further comprises the step of administering said treated cell to the subject, thereby regulating osteogenesis in the subject.

95. The method of claim 94, wherein said regulating osteogenesis and/or bone repair is stimulating or enhancing osteogenesis and/or bone repair, and whereas said modulating said expression and/or said activity of TAK1 is diminishing or abrogating said expression and/or said activity of TAK1.

96. The method of claim 94, wherein said agent comprises a polypeptide encoded by a nucleic acid having a nucleotide sequence at least 70% homologous to SEQ ID NO: 1 and/or 2.

97. The method of claim 94, wherein said agent comprises a single-stranded or double-stranded oligonucleotide which is at least 12 nucleotides in length and is specifically hybridizable with SEQ ID NO: 1 and/or 2.

98. The method of claim 94, wherein said agent comprises an oligonucleotide having a nucleic acid sequence at least 70% homologous to SEQ ID NO: 3 and/or 4.

99. The method of claim 94, wherein said cell with osteogenic potential is selected from the group consisting of a mesenchymal stem cell, a progenitor cell, an osteoblast and a cell capable of differentiating into an osteoblast.

100. The method of claim 94, wherein said cell with osteogenic potential is located in the subject at a site of inflammation, and/or wherein said administering said cell is effected by administering said cell to the subject at a site of inflammation.

101. The method of claim 94, wherein the subject suffers from a disease selected from the group consisting of inflammation-mediated bone loss, periodontal disease, osteoarthritis, Kohler's bone disease, rheumatoid arthritis and osteoporosis.

102. The method of claim 94, wherein said activity of TAK1 is a kinase activity and/or an interaction of TAK1 with an MH2 domain of a SMAD protein.

103. The method of claim 94, wherein said regulating osteogenesis and/or bone repair is diminishing or abrogating osteogenesis and/or bone repair, and whereas said modulating said expression and/or said activity of TAK1 is stimulating or enhancing said expression and/or said activity of TAK1.

104. The method of claim 94, wherein said cell with osteogenic potential is located at a site of lung injury and/or persistent infection in the subject.

105. A composition comprising an isolated nucleic acid having a nucleic acid sequence at least 70% homologous to a nucleic acid sequence of a nucleic acid selected from the group consisting of SEQ ID NO: 1, SEQ ID NO: 2, an antisense strand of SEQ ID NO: 1, and an antisense strand of SEQ ID NO: 2.

106. A vector comprising the nucleic acid sequence of claim 105.

107. The vector of claim 20, further comprising a promoter for regulating transcription of the nucleic acid in sense or antisense orientation, and/or further comprising positive and/or negative selection markers for selecting for homologous recombination events.

108. A host cell or an animal comprising the vector of claim 106.

109. The host cell of claim 108, wherein the host cell is selected from the group consisting of a mesenchymal stem cell, a progenitor cell, an osteoblast and a cell capable of differentiating into an osteoblast.

110. A single-stranded or double-stranded oligonucleotide at least 12 bases in length specifically hybridizable with a nucleic acid selected from the group consisting of SEQ ID NO: 1, SEQ ID NO: 2, an antisense strand of SEQ ID NO: 1 and an antisense strand of SEQ ID NO: 2.

111. The oligonucleotide of claim 110, wherein said oligonucleotide comprises a nucleic acid having a sequence at least 70% homologous to SEQ ID NO: 3 and/or 4.


SEQUENCE LISTING (10 sequences)

SEQ ID NO: 1 - DNA, 911 bp, Mus musculus
atgtcgacagcctccgccgcctcgtcctcctcctcgtcttctgccagtgagatgatcgaagcgccgtcgcaggtcctgaacttcgaagagatcgactacaaggagatcgaggtggaagaggttgtcggaagaggagcttttggagtagtttgcaaagctaagtggagagcaaaagatgtcgctattaaacagatagaaagtgagtctgagaggaaggctttcattgtggagctccggcagttgtcacgtgtgaaccatcctaacattgtcaagttgtatggagcctgcctgaatccagtatgtcttgtgatggaatatgcagaggggggctcattgtataatgtgctgcatggtgctgaaccattgccttactacactgctgctcatgccatgagctggtgtttacagtgttcccaaggagtggcttacctgcacagcatgcagcccaaagcgctgattcacagggacctcaagcctccaaacttgctgctggttgcaggagggacagttctaaaaatctgcgattttggtacagcttgtgacatccaaacacacatgaccaataataaagggagtgctgcttggatggcgcctgaagtatttgaaggtagcaattacagtgaaaagtgtgatgtcttcagctggggtattatcctctgggaagtgataacacgccggaaacccttcgatgagatcggtggcccagctttcagaatcatgtgggctgttcataatggcactcgaccaccactgatcaaaaatttacctaagcccattgagagcttgatgacacgctgttggtctaaggacccatctcagcgcccttcaatggaggaaattgtgaaaataatgactcacttgatgcggtacttcccaggagcggatgagccgttacagtatccttgtcagta

SEQ ID NO: 2 - DNA, 910 bp, Mus musculus
ctctgatgaagggcagagcaactcagccaccagcacaggctcattcatggacattgcttctacaaataccagtaataaaagtgacacaaatatggaacaggttcctgccacaaacgacactattaaacgcttggagtcaaaacttttgaaaaaccaggcaaagcaacagagtgaatctggacgcctgagcttgggagcctctcgtgggagcagtgtggagagcttgccccccacttccgagggcaagaggatgagtgctgacatgtctgaaatagaagccaggatcgtggcgactgcagcctattccaagcctaaacggggccaccgtaaaaccgcttcatttggcaacattctggatgtccctgagatcgtcatatcaggtaacgggcaaccaaggcgtagatccatccaagacttgactgttactgggacagaacctggtcaggtgagcagccggtcatccagccctagtgtcagaatgatcactacctcaggaccaacctcagagaagccagctcgcagtcacccgtggacccctgatgattccacagataccaatggctcagataactccatcccaatggcgtatcttacactggatcaccagctacagcctctagcgccgtgcccaaactccaaagaatccatggcagtgttcgaacaacattgtaaaatggcacaggagtatatgaaagttcaaaccgaaatcgcattgttactacagagaaagcaagaactagttgcagaattggaccaggatgaaaaggaccagcaaaatacatctcgtctggtacaggaacataaaaagcttttagatgaaaacaaaagcctttctacttattaccagcaatgcaaaaaacaactagaggtcatcagaagccaacagcagaaacgacaaggcacttcatga

SEQ ID NO: 3 - DNA, 60 bp, Artificial sequence
  Single strand DNA oligonucleotide
tataggatcctcatcacttgtcatcgtcatccttgtagtcatactgtaatggctcatccg

SEQ ID NO: 4 - DNA, 39 bp, Artificial sequence
  Single strand DNA oligonucleotide
tatagaattcgccaccatgccttgtcagtactctgatga

SEQ ID NO: 5 - DNA, 33 bp, Artificial sequence
  Single strand DNA oligonucleotide
tatagaattccgcgggggatcatgtcgacagcc

SEQ ID NO: 6 - DNA, 70 bp, Artificial sequence
  Single strand DNA oligonucleotide
tataggatcctcatcacagatcctcttctgagatgagtttttgttctgaagtgccttgtcgtttctgctg

SEQ ID NO: 7 - DNA, 21 bp, Artificial sequence
  Single strand DNA oligonucleotide
caactcagccaccagcacagg

SEQ ID NO: 8 - DNA, 21 bp, Artificial sequence
  Single strand DNA oligonucleotide
gactgcgagctggcttctctg

SEQ ID NO: 9 - DNA, 40 bp, Artificial sequence
  Single strand DNA oligonucleotide
tatagaattcgccaccatgtcgacagcctccgccgcctcg

SEQ ID NO: 10 - DNA, 64 bp, Artificial sequence
  Single strand DNA oligonucleotide
tataggatcctcatcacttgtcatcgtcatccttgtagtctgaagtgccttgtcgtttctgctg